(19) United States
(12) Patent Application Publication
(10) Pub. No.: US 2010/0034779 A1
(43) Pub. Date: Feb. 11, 2010

(54) COMPOSITIONS AND METHODS FOR PRODUCING PLURIPOTENT CELLS FROM ADULT TESTIS

(76) Inventors: Kaomei Guan, Waake (DE); Gerd Hasenfuss, Gottingen (DE); Karim Nayernia, Gottingen (DE); Wolfgang Engel, Lenglern (DE)

Correspondence Address: LANDO & ANASTASI, LLP, ONE MAIN STREET, SUITE 1100, CAMBRIDGE, MA, 02142, US

(21) Appl. No.: 12/092,547

(22) PCT Filed: Nov. 2, 2006
(86) PCT No.: PCT/EP06/10529

§ 371 Date: Mar. 11, 2009

Related U.S. Application Data
(60) Provisional application No. 60/732,132, filed on Nov. 2, 2005.

(51) Int. Cl. A61K 45/00; C12N 5/073; C12N 5/071; A01N 1/00; A61P 25/28; A61P 5/00

(52) U.S. Cl. 424/93.7; 435/325; 435/366; 435/363; 435/353; 435/354; 435/352; 435/350; 435/1.1

(57) ABSTRACT

The present application describes a method of producing embryonic stem cell (ESC)-like cells derived from adult mammalian testis. Furthermore, the application describes to a method of producing embryoid bodies from ESC-like cells as well as a method of producing a tissue and/or a differentiated cell from the ESC-like cell or the embryoid body. In addition, an ESC-like cell, an embryoid body and/or differentiated cell and/or tissue obtainable by said methods and pharmaceutical preparations containing the same are provided. Finally, the application describes to the use of these products for medical treatments and the preparation of pharmaceutical compositions for medical treatments.

### FIELD OF THE INVENTION

The invention relates to the isolation and culture of adult stem cells. More specifically, the invention relates to adult spermatogonial stem cells as well as the culture of embryonic stem cell-like cells (ESCs), embryoid bodies, and differentiated cells or tissues derived from adult spermatogonial stem cells.

### BACKGROUND OF THE INVENTION

In the mouse, the embryonic precursors of the gametes, known as primordial germ cells (PGCs), arising from embryonal ectoderm are identified in the embryo around day 7 post-coitum. Over the next few days, they proliferate during migration and reach the genital ridges. In males, they enter foetal seminiferous tubules, become gonocytes, and cease dividing. Following birth in mammals, gonocytes migrate to the seminiferous tubule basement membrane and develop to type A spermatogonia, the spermatogonial stem cells (SSCs). SSCs have the capability to self-renew and to produce large numbers of progenitors that are destined to differentiate into spermatozoa throughout life (Spradling, A. et al., Nature 414, 2001, 98-104). Embryonic germ cells (EGCs) derived from PGCs are pluripotent and have differentiation potential similar to embryonic stem cells (ESCs) (Resnick, J. L. et al., Nature 359, 1992, 550-1; Matsui, Y. et al., Cell 70, 1992, 841-7.). Germline stem cells from neonatal (0-2 days old) mouse testis are also pluripotent and able to differentiate into various types of somatic cells (Kanatsu-Shinohara, M. et al., Cell 119, 2004, 1001-12) suggesting that the germline lineage may retain the ability to generate pluripotent cells. However, until now there has been no evidence for the pluripotency and plasticity of adult spermatogonial stem cells (SSCs), which are responsible for maintaining spermatogenesis throughout life in the male (Spradling, 2001, supra).

In a previous study by Kanatsu-Shinohara et al. (2004, supra) pluripotent ES-like cells expressing the c-kit antigen were successfully generated from neonatal mouse testis and induced to differentiate further into various tissue types. The authors also attempted to obtain ES-like cells from an enriched preparation of adult spermatogonial stem cells derived from wildtype mouse testis, but no ES-like cells could be obtained under the culture conditions used therein. However, the authors could obtain ES-like cells in two of eight experiments from p53 knockout mice. Since knockout of p53 results in enhanced tumorigenesis (Donehower, L. A., et al., Mol Carcinog 1995, 14, 16-22), and the cells are not characterized by Kanatsu-Shinohara et al. (2004, supra), the origin of the ES-like cells is not clear.

U.S. Pat. No. 6,251,671 (Hogan et al.) suggests a method of making a population of mammalian pluripotent embryonic stem cells by culturing neonatal or postnatal SSCs either in the presence of a culture medium containing BMP8 protein and/or growth enhancing amounts of basic fibroblast growth factor (bFGF), leukemia inhibitory factor (LIF), and membrane associated and soluble steel factors, the latter constituting ligands for the c-kit receptor. However, no further characterization of cells obtained in such a way is shown, and no method of further differentiating such cells into embryoid bodies and/or differentiated cells is mentioned in the document.

Pluripotent ESC-like cells obtained from adult spermatogonial stem cells may have great potential for cell-based organ regeneration therapy due to their origin from adult tissue. Since these cells are pluripotent like ESCs, use of their differentiated derivatives for autologous transplantation may allow establishment of individual cell-based therapy without the ethical problems associated with human ESCs. In addition to cell transplantation strategies, use of ESC-like cells derived from SSCs of transgenic animals or from testicular biopsies of humans with genetic abnormalities or varieties might allow developing various cell types for studying physiological or pathophysiological processes under in vitro conditions.

One object of the present invention therefore is to establish new cell culture conditions to which isolated adult spermatogonial stem cells (SSCs) respond in a reproducible manner and acquire embryonic stem cell (ESC)-like properties such as the ability to differentiate spontaneously into derivatives of three embryonic germ layers in vitro and in vivo. Culture conditions for the initial culture of testicular cells used until now included subjecting the cells to a variety of growth factors which may result in the overgrowth of other cells, including fibroblasts, endothelial and Sertoli cells.

Another object of the present invention is to provide ESC-like cells and embryoid bodies derived from adult spermatogonial stem cells for producing a tissue or a differentiated cell that may be used in cell-based organ-regeneration therapy and autologous or allogenic cell, tissue, or organ transplantation.

### SUMMARY OF THE INVENTION

Accordingly, the present invention relates to a method of producing embryonic stem cell (ESC)-like cells derived from adult mammalian testis, as defined in the claims. Furthermore, the invention relates to a method of producing embryoid bodies from ESC-like cells as well as a method of producing a tissue and/or a differentiated cell from the ESC-like cell or the embryoid body, as defined in the claims. In addition, an ESC-like cell, an embryoid body and/or differentiated cell and/or tissue obtainable by said methods and pharmaceutical preparations containing the same are provided, as defined in the claims. Finally, the invention relates to the use of these products for medical treatments and the preparation of pharmaceutical compositions for medical treatment, respectively, as specified in the claims.

### DETAILED DESCRIPTION OF THE INVENTION

The present invention accomplishes the establishment of cell culture conditions containing only the growth factors and feeder cells essential for the successful proliferation of adult SSCs, and it was surprisingly found that thus cultured SSCs can develop into ESC-like cells and further differentiated cell and tissue forms.

In a first aspect, the present invention relates to a method of producing embryonic stem cell (ESC)-like cells derived from adult mammalian testis, comprising the steps of (a) propagating isolated mammalian testicular cells (i) in a suitable culture medium containing serum or serum replacement and optionally containing one or more growth factors selected from the group consisting of leukaemia inhibitory factor (LIF), glial cell line-derived neurotrophic factor (GDNF), basic fibroblast growth factor (bFGF), (2′Z, 3′E)-6-Bromoindirubin-3′-oxime (BIO; GSK-3 inhibitor IX) and B27, and/or (ii) in a suitable culture medium containing serum or serum replacement on a suitable feeder layer; for a period sufficient to allow the formation of ESC-like cells; and (b) optionally obtaining the ESC-like cells from the culture.

The “ESC-like cells” obtained according to the above method exhibit properties of embryonic stem cells (ESCs). In particular, they may give rise to cells derived from all three embryonic germ layers, i.e. mesoderm, endoderm, and ectoderm and, thus, all cells of the body. The ESC-like cells are derived from adult mammalian testis. In a first step, isolated mammalian testicular cells are propagated in a suitable culture medium. The testicular cells can be obtained for example from testis biopsies and may be isolated as described in the experimental section herein. Further methods for the isolation of such cells are known to the skilled person (see, e.g., U.S. Pat. No. 6,251,671).

The thus isolated testicular cells are then transferred in a suitable cell culture medium for continued growth and expansion. Typically, the cells are further passaged upon reaching about 40 to 70% confluency. Usually, cells from human, non-human primate and mouse are passaged every 1-7 days. ESC-like cells can be obtained after 2 to 3 passages in case of murine cells, and after 5-7 passages in case of human or non-human primate cells. In principle, it is possible to culture these ESC-like cells up to 30 passages and longer.

By monitoring the appearance of tightly packed ESC-like colonies, as those shown in the appended drawings, the skilled person can readily determine whether a sufficient number of cells have been obtained so that the propagating may be terminated. Methods to retrieve single ESC-like colonies include picking them by micromanipulation from the culture under microscope supervision. It is, in principle, possible to culture these ESC-like cells up to 30 passages and longer.

Typical examples of suitable culture mediums are commercially available DMEM, DMEM/F12 and KnockOut™ D-MEM (GIBCO®) media. For human or non-human primate cells DMEM/F12 medium is generally used, whereas for mouse cells DMEM is typically used. Alternative media are available to the skilled person from various commercial sources and can be tested for their positive effect on the growth of the desired cells. The medium may be supplemented with serum or serum replacement. The serum may be Fetal Calf Serum (FCS), or an equivalent serum derived from other species such as human, horse and pig. Alternatively, a commercially available synthetic serum replacement such as KnockOut™ Serum Replacement (GIBCO®) may be used. The serum or serum replacement may be contained in the respective medium in a quantity of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30%, in particular 5, 10, 15, 20 and 25% (v/v). The medium may also contain additional additives such as glutamine, non essential amino acids, β-mercaptoethanol, and antibiotics, all of which are known to the skilled person and can, thus, be optimized in accordance with standard procedures.

In addition, the medium may contain one or more growth factors, supporting the growth of the desired ESC-like cells. Typical examples are leukaemia inhibitory factor (LIF), glial cell line-derived neurotrophic factor (GDNF), BIO and B27, which are commercially available from a variety of distributors. Proliferating-enhancing amounts of these factors can vary depending on the species or strain of the cells, and the type of purity of the factors. Generally, 0.5 to 500 ng/ml of each factor within the culture solution is appropriate. In particular, the amount is between 4-50 ng/ml for GDNF, 1-10×10³ units/ml for LIF, 0.5-5 μM BIO, and 1×B27. If basic fibroblast growth factor (bFGF) is used, e.g. for human and non-human primate cells, it is added in a quantity of 1-10 ng/ml. Typical combinations of growth factors are shown in Table 1 below.

Alternatively, or additionally, the culture may take place on a suitable feeder layer. Typical examples of feeder layer cells are embryonic fibroblasts derived from a mammal such as mouse, inactivated e.g. by mitomycin C-treatment or irradiation. Other conventional feeder layers may also be used, if desirable.

Suitable containers used for the culture of these cells may include, but are not limited to, conventional culture dishes, uncoated or coated with e.g. gelatine or Matrigel.

Optionally, the cells can be obtained from the culture by dissociating them with a suitable concentration of e.g. trypsin/EDTA or Collagenase IV, or any other commercially available substance such as TrypLE™ Select or TrypLE™ Express (Invitrogen) or Akutase or Dispase.

In a preferred embodiment, at most 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, in particular at most 30%, more particular at most 10%, of the ESC-like cells exhibit the c-kit antigen. The determination of the c-kit antigen expression may be performed using c-kit-specific antibodies in conventional FACS sorting in accordance with Examples 3.1 (mouse), 3.2 (human and non-human primate) given herein. The c-kit receptor (also known as CD117) is known to be expressed in differentiating spermatogonia and is listed, for example, in (Yoshinaga, K., et al., Development 113, 1991, 689-99; Vincent, S., et al., Development 125, 1998, 4585-93) under Accession Number NM021099; see also U.S. Pat. No. 6,949,364.

In a preferred embodiment, the mammalian testis cells are selected from the group consisting of human, non-human primate, mouse, bovine, pig, goat, rat, rabbit, ovine, horse, dog, cat and guinea-pig cells. The inventors show in the present application that they were able to successfully obtain ESC-like cells and products derived from these from three different species, i.e. human, non-human primate and mouse. Therefore, it is contemplated that the skilled person may also obtain similar results when using cells from other species such as listed above.

In a preferred embodiment, human or non-human primate cells are kept in a medium such as described above. Usually, the medium additionally contains one of the growth factors listed in Table 1 or any of the combinations of growth factors shown in the same table. In particular, all five of the growth factors listed in Table 1 are added to the medium. This medium may also be used in combination with a suitable feeder layer as defined above. Furthermore, when non-human primate cells are cultured, the medium may be DMEM further containing serum or serum replacement, as described above, and LIF and/or a feeder layer, as described above.

TABLE 1
Combinations of growth factors

| | GDNF | LIF | bFGF | BIO | B27 |
|---|---|---|---|---|---|
| 1 | x | | | | |
| 2 | | x | | | |
| 3 | | | x | | |
| 4 | | | | x | |
| 5 | | | | | x |
| 6 | x | x | | | |
| 7 | x | | x | | |
| 8 | x | | | x | |
| 9 | x | | | | x |
| 10 | | x | x | | |
| 11 | | x | | x | |
| 12 | | x | | | x |
| 13 | | | x | x | |
| 14 | | | x | | x |
| 15 | x | x | x | | |
| 16 | x | x | | x | |
| 17 | x | x | | | x |
| 18 | x | | x | x | |
| 19 | x | | x | | x |
| 20 | | x | x | x | |
| 21 | | x | x | | x |
| 22 | | x | | x | x |
| 23 | | | x | x | x |
| 24 | x | x | x | x | |
| 25 | x | x | x | | x |
| 26 | x | x | | x | x |
| 27 | x | | x | x | x |
| 28 | | x | x | x | x |
| 29 | x | x | x | x | x |

In another embodiment, the mammalian cells are of murine origin. Typically, the medium used herein is DMEM containing serum or serum replacement, as described above, particularly in the presence of LIF and/or a feeder layer, as described above. Preferably, the propagating takes place in the substantial absence of bFGF, EGF, and/or GDNF, in particular in the substantial absence of bFGF or EGF within the first 14 days, more particularly the first 7 days, most particularly the first 2-4 days of culture of the isolated mammalian testis cells. Thus, GDNF may be present during the first 1, 2, 3, 4, 5, 6, 7, 8, 9, 10, 11, 12, 13, or 14 days of culture. The term “substantial absence”, as used herein, refers to a quantity of less than 10, 9, 8, 7, 6, 5, 4, 3, 2, 1 ng/ml bFGF or zero bFGF, less than 20, 19, 18, 17, 16, 15, 14, 13, 12, 11, 10, 9, 8, 7, 6, 5, 4, 3, 2, 1 ng/ml EGF or zero EGF, and/or less than 10, 9, 8, 7, 6, 5, 4, 3, 2, 1 ng/ml GDNF or zero GDNF, in particular less than 0.5 ng/ml of each factor.

If desired, the propagating of the mammalian testicular cells takes place in the substantial absence of steel factor (SF) and/or Bone Morphogenic Protein 8 (BMP8). In particular, SF and/or BMP8 are present in a concentration of less than 100, 90, 80, 70, 60, 50, 40, 30, 20, 15, 12, 5, 10, 9, 8, 7, 6, 5, 4, 3, 2, 1, 0.5 ng/ml or zero SF and/or BMP8, more particularly less than 0.5 ng/ml of culture medium. The use of SF and BMP8 is described in U.S. Pat. No. 6,251,671. SF is a ligand to the c-kit receptor and is known to induce the differentiation of SSCs into meiotic and post-meiotic cells (Feng, L. X. et al., Science 2002, 297, 392-395). In another embodiment, prior to step a) of claim 1, the number of the isolated mammalian testicular cells can be increased, if desired, by propagating the cells in a suitable cell culture medium containing serum or serum replacement as described above, for a period of 20, 19, 18, 17, 16, 15, 14, 13, 12, 11, 10, 9, 8, 7, 6, 5, 4, 3, 2, or 1 days. This step may be performed to further improve the quality and number of the isolated mammalian testicular cells, in particular if the testis biopsy should not provide sufficient material. Ideally, the step is carried out until SSC-colonies are observable in the culture which the skilled person will recognize as having reached a suitable size and number. As an example, suitable colonies may contain 20-100 cells and have a diameter of about 60-250 μm.

In another embodiment, the mammalian ESC-like cells are expanded after step (a) and before step (b) in the presence of a suitable feeder layer as described above and/or one or more suitable growth factors as described above for a period of 1-250, in particular 15-210, more particularly 30-210, most particularly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 days. Usually, the ESC-like cells are passaged at a dilution of 1:3 to 1:6, typically every 1-4 or 1-7 days.

In a further embodiment, the mammalian testicular cells are provided as a preparation derived from an adult mammalian testis as described above. The preparation may be enriched for spermatogonial stem cells (SSCs) for example by a) selecting cells according to protein expression in the same cells or by b) use of a pre-plating method or by c) mechanical isolation of ESC-like colonies from the culture under microscope supervision. If step a) is chosen, examples for such proteins suitable for selection are surface proteins such as CD-9 or SSEA1 for murine cells, EpCAM and SSEA3 for human cells, or Integrin α6 or Integrin β1. The expression of such proteins may be monitored for example by antibody staining and FACS sorting. Another technique is the selection of cells by promoter-driven gene expression as described in example 1.1 below. An example for a pre-plating method as mentioned under b) above is also described among others in the examples 3.1, 5.1 following below. Such enrichment techniques have the potential to increase the number of SSCs obtained by 5-fold to 700-fold.

In another embodiment, the mammalian ESC-like cells express different levels of the antigens c-kit, Thy-1 and/or the gene Stra8. Sequences for the Thy-1 protein and the Stra8 gene can be found under Accession Number BC065559 for Thy-1 and NM 009292 for Stra8. The expression levels of the antigens c-kit and Thy-1 may be determined by techniques such as FACS sorting as described in the examples 3.1 and 3.2 described below. Typical expression levels of the antigens c-kit and Thy-1 found in ESC-like cells of three different species are displayed in the following Table 2. In a preferred embodiment, the ESC-like cells are characterized by the expression of Stra8 and/or c-kit. The c-kit expression may be exhibited by at most 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, in particular at most 30%, more particularly at most 10%, of the ESC-like cells. Expression of the gene Stra8 can be determined by procedures such as standard RT-PCR using suitable oligonucleotide primers such as described in example 4 below and subsequent analysis for example by subjecting the PCR probe to standard agarose gel electrophoresis. Expression of the gene Stra8 can be ascertained by the appearance of a visible band on such a gel.

TABLE 2
Typical percentages of ESC-like cells showing antigen expression
as determined by FACS sorting

| | Human | Non-human primate | Mouse |
|---|---|---|---|
| c-kit | weakly positive | negative | weakly positive |
| | <1-5% | <1% | <1-30% |
| Thy-1 | strongly positive | medium positive | weakly positive |
| | >90% | 45-80% | <1-40% |

In another embodiment, the mammalian ESC-like cells are capable of forming embryoid bodies as defined below.

In another aspect, the present invention relates to an ESC-like cell as obtainable by the process described above and in the exemplifying section. In particular, these cells are characterized by the expression of the gene Stra8 and/or the c-kit receptor, as outlined above.

In still another embodiment, the invention relates to a pharmaceutical preparation comprising the ESC-like cells and a pharmaceutically acceptable carrier. These pharmaceutical preparations contain ESC-like cells according to the invention in a quantity of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50% particularly 60 or 70%, more particularly 80 or 90% and most particularly 100%, relative to the total number of cells present in the preparation.

Another aspect of the invention relates to a method of producing embryoid bodies from ESC-like cells, comprising culturing the ESC-like cells according to the invention under conditions effective to yield embryoid bodies, and optionally recovering the embryoid bodies from the culture. The term “embryoid body”, as used herein, refers to clumps of cellular structures that contain tissue from all three germ layers: endoderm, mesoderm, and ectoderm. The typical morphology of embryoid bodies is shown in FIG. 5. Conditions effective to yield embryoid bodies allow to reliably producing these embryoid bodies. Typical time periods for the appearance of embryoid bodies derived from ESC-like cells are 2-5 days for cells of murine origin, and 2-7 days for cells of human or non-human primate origin. Single embryoid bodies can be isolated for example by picking them from the culture dish with the help of a pipette tip. Examples for conditions effective to yield embryoid bodies are provided by applying the so-called hanging drop technique and the mass culture, as described in examples 5.1 and 5.2. Additional methods are the methylcellulose (Wiles, M V and Keller, G., Development 1991, 111, 259-67), and the spinner-culture method (Zandstra, P W et al., Tissue Eng. 2003, 9, 767-78; Gerecht-Nir, S. et al., Biotechnology and Bioengineering Early View Online: 8 Apr., 2004), which is particularly advantageous for an industrial setting. Other methods for in vitro differentiation of ESC-like cells include the cultivation of these cells on stromal feeder layer like OP9 as described in Nakano T. et al, Science 1994, 265, 1098-1101. In the light of the present disclosure, it is contemplated that the skilled person can readily obtain embryoid bodies from ESC-like cells derived from the different species referred to herein. The present disclosure thus provides embryoid bodies obtainable by the process according to the invention.

In another embodiment, the invention relates to a pharmaceutical preparation comprising an embryoid body according to the invention and a pharmaceutically acceptable carrier.

In still another aspect the present invention relates to a method of producing a tissue and/or a differentiated cell, comprising culturing an ESC-like cell according to the invention and/or an embryoid body according to the invention under conditions effective to yield the tissue and/or the differentiated cell, and optionally recovering the tissue and/or the cell. For example, cardiomyocytes may be obtained via embryoid bodies, while neuronal cells may be obtainable directly from the ESC-like cell according to the invention. Depending on the desired cell or tissue type, the skilled person will select the appropriate factors and culture conditions suitable for obtaining the desired product as well as suitable tissue or cell-specific markers to verify that the desired products have been obtained. Examples for such tissue-specific markers are provided in the experimental section below in 5.1. In addition, it is contemplated that tissue-specific promoters driving a gene encoding for example antibiotics or fluorescent proteins are introduced by transfection of the cell culture and the desired differentiated cells are then selected by way of, e.g. FACS sorting or antibiotic selection. Examples for such promoters include the isl-1, Nkx2.5, MLC-2v or MHC promoter for the selection of cardiomyocytes, the promoters of myoD or myf5 for selection of myocytes, the promoter of α-fetoprotein or albumin for selection of hepatocytes, the promoter of nestin for selection of neural progenitor cells, the promoter of TH for selection of dopaminergic neurons, or the promoter of Nkx2.2, insulin or Nkx6.1 for selection of pancreatic cells. Methods of recovering cells from the culture include for example a mechanical selection such as shown in 5.1 of the experimental section.

In a preferred embodiment the desired tissue is selected from the group consisting of heart, vessels, neurons, skin, the eye, the nose, the ear, the spinal cord, a nerve, the trachea, the mouth, the esophagus, the small intestine, the large intestines, the ureter, the bladder, the urethra, a gland such as hypothalamus, pituitary, thyroid, pancreas and adrenal glands, the ovary, the oviduct, the uterus, the vagina, a mammary gland, the testes, the penis, a lymph node, a tendon, a ligament, brain, intestine, lung, muscle, stomach, liver, kidney, spleen, fat, bone, cartilage and epithelium. In another embodiment, the cell is selected from the group consisting of a cardiomyocyte, a skeletal muscle cell, a smooth muscle cell, an endothelial cell, a vascular cell, a vascular smooth muscle cell; a neural cell, a neuronal cell, in particular a neuron, more particularly a dopaminergic neuron, a cholinergic neuron, a GABAergic neuron, a serotoninergic neuron; a glial cell, a dendritic cell, a hepatocyte, a pancreatic cell, particularly a pancreatic hormone-producing cell; more particularly an insulin-expressing cell, a beta-cell, a glucagon-expressing cell, an alpha-cell, a somatostatin-expressing cell, a pancreatic polypeptide-expressing cell; a chondroicyte, a blastocyte, a astrocyte, a oligodendrocyte, a blood cell, a progenitor cell, a urogenital cell, a gastrointestinal cell, a glandular cell, an adiopocyte, an osteocyte, a microglia, an epithelial or epitheloid cell, and a bile duct cell.

In still a further embodiment the invention relates to a tissue and/or differentiated cell thus obtained. As set out above, the type of tissue and/or cell can be verified by use of tissue and/or cell-specific markers such as those identified in the experimental section herein below.

The invention also relates to a pharmaceutical preparation comprising a cell and/or tissue as outlined above and a pharmaceutically acceptable carrier. These pharmaceutical preparations contain cells or tissues according to the invention in a quantity of at least 1, 2, 3, 4, 5, 6, 7, 8, 9, 10, 11, 12, 13, 14, 15, 16, 17, 18, 19, 20, 21, 22, 23, 24, 25, 26, 277, 28, 29, 30, 31, 32, 33, 34, 35, 36, 37, 38, 39, 40, 41, 42, 43, 44, 45, 46, 47, 48, 49, 50% particularly 60 or 70%, more particularly 80 or 90% and most particularly 100%, relative to the total number of cells present in the preparation.

In another aspect, the invention relates to the use of an ESC-like cell, an embryoid body, or a tissue or cell, or a pharmaceutical composition as outlines above for producing cell lines, tissues, organs, blastocysts, and transgenic animals. Examples for ways to obtain such cell lines are described in the examples below. It is apparent that such cell lines might be useful for studying diseases. The establishment of cell lines from humans or animals with genetic abnormalities or varieties could provide new options for studying the corresponding cellular phenotype under in vitro conditions. Examples for the use of blastocysts and the creation of transgenic animals are also provided in the examples below in 6.1 and 6.2 of the experimental section. It is contemplated that such transgenic animals would allow the development of various cell types for studying physiological or pathopysiological processes. In the light of present disclosure the skilled person can readily produce such transgenics.

In another embodiment, the use of an ESC-like cell, an embryoid body, a tissue or cell, or any of the pharmaceutical compositions described above is contemplated for the use in cell-based organ regeneration therapy, tissue-based organ regeneration therapy, and autologous or allogenic cell, tissue or organ transplantation. Such uses might allow the establishment of individual cell-based therapy, since the donor and recipient of the cell and/or tissue could be identical. The immunological profile of these cells might allow also allogenic transplantation. It is evident that by the use of such techniques, the ethical problems associated with human embryonic stem cells might be avoided.

In another embodiment, the use of an ESC-like cell, an embryoid body, a tissue or cell, or any of the pharmaceutical compositions outlined above is contemplated for the prevention and/or therapy of a disease or disorder such as chronic heart disease, Parkinson's disease, diabetes, liver failure, infertility and end-stage kidney disease, traumatic spinal cord injury, Purkinje cell degeneration, neural degeneration, Alzheimer's disease, Lewy Body Dementia, multiple sclerosis, duchenne's muscular dystrophy, muscular dystrophy, heart failure, osteogenesis imperfecta, osteoporosis, cancer, an autoimmune disease, a neurodegenerative disease, a respiratory disease, a vascular disease, a trauma, burn, head trauma, spinal cord injury, stroke, myocardial infarction, arthrosis, Huntington's disease, Tourette's syndrome, amytrophic lateral sclerosis, Addison's disease, pituitary insufficiency, liver failure, inflammatory arthrophathy, neuropathic pain, blindness, hearing loss, arthritis, a bacterial infection, a viral infection, a sexually transmitted disease and a damage of the skin, the eye, the nose, the ear, the brain, the spinal cord, a nerve, the trachea, the lungs, the mouth, the esophagus, the stomach, the liver, the small intestines, the large intestines, the kidney, the ureter, the bladder, the urethra, a gland such as hypothalamus, pituitary, thyroid, pancreas and adrenal glands, the ovary, the oviduct, the uterus, the vagina, a mammary gland, the testes, the penis, a lymph node, a vessel, the heart, a blood vessel, a skeletal muscle, a smooth muscle, a bone, cartilage, a tendon or a ligament. It is contemplated that, based on the present disclosure, the skilled person may readily use the ESC-like cells, embryoid bodies or pharmaceutical compositions of the invention to put the above-described medical applications into practice.

### DESCRIPTION OF THE FIGURES

FIG. 1. Cellular and molecular characterisation of cultured mouse SSCs and maGSCs. a, Epiblast-like colony under culture condition I. b, ES-like colonies appeared under culture condition II. c, A typical colony of established culture in condition IV at passage 30. Scale bar, 50 μm. d-g, Double immunostaining of maGSCs by antibodies against GFP (green, d, e, g) and the cell surface marker stage-specific embryonic antigen (SSEA-1) (red, e), the germline-specific transcription factor Oct4 (red, f), and SSEA-3 (red, g). Scale bar, 25 μm. h, i, Staining for alkaline phosphatase (ALP). SSCs cultured under condition IV (=maGSCs; h) are strongly positive, whereas SSCs under condition II (i) are only partially positive. Scale bar, 50 μm. j, k, RT-PCR analyses of transcription factors essential for undifferentiated cells in SSCs cultured under conditions I, II, III, and IV (j) and during differentiation of EBs after plating at day 5 (d5; k). FL, feeder layer of mouse embryonic fibroblasts. M, DNA marker.

FIG. 2. a, ESC-like colonies derived from testicular cells isolated from C37BL/6 mouse. Scale bar, 100 μm. b, RT-PCR analyses of transcription factors typical of undifferentiated cells in three cell lines derived from C57BL/6, 129/Ola and FVB mice separately when cultured under conditions III and IV. FL, feeder layer of mouse embryonic fibroblasts. M, 100 bp DNA markers.

FIG. 3. a, b, ES-like colonies derived from non-human primate SSCs appeared under culture condition II (a) and III (b) after 4-5 passages. c, ES-like colonies derived from non-human primate SSCs appeared when culturing cells in DMEM/F12 medium containing 15% FCS, 2 mM glutamine, 1×NEAA, 50 μM β-ME, LIF (10³ Units/ml) and BIO (1 μM) after 2-weeks. d, e, ES-like colonies derived from human SSCs appeared when culturing cells on feeder layer in DMEM/F12 medium containing 15% FCS, 2 mM glutamine, 1×NEAA, 50 μM β-ME, GDNF (10 ng/ml), bFGF (2 ng/ml), LIF (10³ Units/ml) and 1×B27 after 7 passages. f, ES-like colonies derived from human SSCs appeared also when cells are cultured on feeder layer in DMEM/F12 medium containing 15% FCS, 2 mM glutamine, 1×NEAA, 50 μM β-ME, LIF (10³ Units/ml) and BIO (1 μM) after 5 passages. Scale bar, 50 μm. g, h, RT-PCR analyses of transcription factors typical of undifferentiated cells in cultured non-human primate SSCs (g, cells are from two different monkeys: A1 and T2) under conditions I and II at passage 3 as well as human SSCs (h, cells are from 3 different donors: Hu1, Hu2 and Nano1) at passages 10 (Hu1), 9 (Hu2), 5 (Nano1, 4 and 5 left) and 15 (Nano1, 5 right and 6) under conditions 1-5.1, Cells were cultured on feeder layer in DMEM/F12 medium containing 20% FCS, 2 mM glutamine, 1×NEAA and 50 μM β-ME. 2, Cells were cultured on feeder layer in DMEM/F12 medium containing 5% FCS, 5% KOSR, 2 mM glutamine, 1×NEAA, 50 μM, β-ME and BIO (1 μM). 3, Cells were cultured on feeder layer in DMEM/F12 medium containing 15% FCS, 2 mM glutamine, 1×NEAA, 50 μM, β-ME and LIF (10³ Units/ml). 4-6, Cells were cultured on feeder layer in DMEM/F12 medium containing 15% FCS, 2 mM glutamine, 1×NEAA, 50 μM β-ME, 2 ng/ml bFGF and 1×B27; additional 10 ng/ml GDNF (4, 5) and 10³ Units/ml LIF (4) were applied. FL, feeder layer of mouse embryonic fibroblasts. M, DNA marker.

FIG. 4. Phenotypic characteristics of cultured GFP+SSCs and SSC-derived maGSCs by Flow cytometric analysis. a, Flow cytometric analysis of EGFP fluorescence in cultured SSCs. Black line, ESCs as control; red line, EGFP fluorescence of SSCs cultured in conditions I, II, III and IV. b, Flow cytometric characterizations of stem cell-specific makers in cultured SSCs in comparison to mouse ESCs. Similar to mouse ESCs, SSCs cultured in all four conditions (I-IV) were strongly positive for SSEA-1 and Oct4, weakly positive for SSEA-4 and Sca-1, and completely negative for SSEA-3, Ter119 and CD34. SSCs cultured on feeder layers (conditions III and IV) showed lower positive for c-kit than those cultured without feeder layers (Conditions I and II) whereas as SSCs cultured in the presence of LIF (conditions II and IV) revealed lower positive for Thy1 than those cultured without LIF (Conditions I and III). Black line, control immunoglobulin; red line, specific antibodies.

FIG. 5. Phenotypic characteristics of cultured non-human primate (a) and human SSCs (b) by Flow cytometric analysis. a, Non-human primate SSCs cultured in DMEM/F12 medium containing 15% FCS, 2 mM glutamine, 1×NEAA and 50 μM β-ME are partially positive for CD90 (Thy-1), CD9 and CD49f (integrin α6), but negative for CD117 (c-kit) (left panel). When non-human primate SSCs cultured in medium containing additional GDNF (10-50 ng/ml), bFGF (1-10 ng/ml), LIF (10³ Units/ml) and 1×B27, more cells are positive for CD90, CD9 and CD49f (right panel). b, Human SSCs cultured in DMEM/F12 medium containing 15% FCS, 2 mM glutamine, 1×NEAA and 50 μM β-ME are strong positive for CD90, very low positive or negative for CD9, CD49f and CD117 (c-kit) (left panel). When human SSCs cultured in medium containing additional GDNF (10-50 ng/ml), bFGF (1-10 ng/ml), LIF (10³ Units/ml) and 1×B27, almost all cells are negative for CD117 (right panel). Little more cells are positive for CD49f. Black line, control immunoglobulin; red line, specific antibodies.

FIG. 6. a, Morphologies of 2-(left, Scale bar, 100 μm), 5-(middle, Scale bar, 100 μm) and 5+2-(right, Scale bar, 200 μm) day-old EBs derived from murine ESC-like cells using hanging drop methods. b, GFP positive cells in EBs shown in a.

FIG. 7. RT-PCR analysis of lineage-specific transcription factors and genes at different stages during differentiation of EBs after plating at day 5 (d5). M, DNA marker. d0, maGSCs before EB formation. FL, feeder layer.

FIG. 8. Cardiac differentiation of mouse maGSCs. a, Percentage of EBs containing beating cardiac clusters during differentiation. b-d, Double immunostaining, of cardiac clusters by antibodies against sarcomeric myosin heavy chain (b) and cardiac specific L-type calcium channels (c); (d) overlay of b and c. Nuclear staining with DAPI. e, Effect of diltiazem (a calcium channel blocker) and (S)-BayK 8644 (a calcium channel activator) on the contraction rate of cardiomyocytes derived from maGSCs. *P<0.05; **P<0.01; ***P<0.001. Scale bar, 25 μm (b-d).

FIG. 9. Cardiac differentiations of murine maGSCs. a-c, Organisation of sarcomeric proteins α-actinin (a), sarcomeric myosin heavy chain (b), and cardiac troponin T (c) in isolated cardiac myocytes at day 5+7. Scale bar, 25 μm. d-f, Connexin 43 (d, green) in cluster of uninucleate day 5+7 beating cardiac cells stained with sarcomeric α-actinin (e). f, Overlay of d and e. Scale bar, 25 μm. g-i, A cluster of uninucleate beating cardiac cells (day 5+11) stained with sarcomeric α-actinin (g) and pan-cadherin (h); (i) overlay of g and h. Scale bar, 25 μm. Nuclear staining with DAPI.

FIG. 10. Functional gap junctions in murine SSC cell clusters. a-b, Confocal images of SSC cell clusters loaded with the gap junction permeable dye (calcein AM) before (Pre), immediately after photobleaching of a single cell (arrow, 0 min), and after a 5 min recovery interval are shown (scale bar, 10 μm). a, Immediately after photobleaching, the fluorescence intensity of the bleached cell is dramatically decreased whereas adjacent cells remain unaffected. After 5 min recovery, this decrease is almost completely reversed consistent with dye refill from adjacent cells through functional gap junctions. b, In the presence of the gap junction uncoupler carbenoxolone (50 μmol/L), the recovery of fluorescence after photobleaching was almost completely abolished. c-d, Mean data for the magnitude of refill after 30 s recovery (c) and the rate of recovery over 5-10 min recovery interval (k, d). Compared to vehicle, both the dye refill after 30 s recovery (c) as well as the rate of recovery within 5-10 min (d) were significantly reduced in the presence of carbenoxolone.

FIG. 11. Action potential characteristics of spontaneously contracting cardiomyocytes at day 5+9. a, Exemplary original traces of APs are shown. Distinct AP morphologies representing pacemaker-, ventricle-, atrial-, and purkinje-like action potentials could be discriminated. The mean data for C_{M}, APA, APD, max. dV/dt and MDP are presented in Table 3. b, In a subset of cardiomyocytes, an intermediate phenotype between pacemaker- and ventricle-like APs was found. c, Effect of β-adrenergic stimulation with isoproterenol (Iso, 1 μmol/L) on the frequency of spontaneous APs. Lefthand, original trace before and during treatment with Iso. An increase in AP frequency and irregularity was observed. Righthand, mean data are shown. d, Non-specific blockade of voltage-gated L-type Ca²⁺ and Na⁺ channels with cadmium (0.5 mmol/L) completely abolished cardiac APs.

FIG. 12. Spontaneous intracellular Ca²⁺ fluctuations in cardiomyocytes derived from murine maGSCs at day 5+7. a, A typical triangle-shaped cardiomyocyte derived from maGSCs using frame scan mode during low diastolic and high systolic [Ca²⁺]ᵢ. Scale bar, 10 μm. b, Average intracellular [Ca²⁺] amplitudes. Filled bar, n=12 vs. open bar, n=5 (P=not significant). c, Line scan mode and corresponding 2D-graph to assess rhythmic Ca²⁺ transients. d, Line scan mode and corresponding 2D-graph detecting elementary Ca²⁺ release events (white bar; Ca²⁺ sparks).

FIG. 13. Expression of calcium related proteins. a, RT-PCR analysis of genes encoding RyR2, PLB, SERCA2a, NCX1 and calsquestin (CASQ) during differentiation of EBs after plating at day 5 (d5). M, DNA marker. d0, maGSCs before EB formation. FL, feeder layer. b-d, RyR2 (b, red), NCX1 (c, red) and SERCA2a (d, red) staining in cluster of uninucleate day 5+7 beating cardiac cells stained with sarcomeric α-actinin (b-d, inset, green). Scale bar, 25 μm. Nuclear staining with DAPI.

FIG. 14. a, Nebulin-positive myotubes differentiated from murine maGSCs at differentiation day 5+23. Scale bar, 25 μm. b-e, DiI-acLDL uptake (b, red) and lectin binding (c, green) of endothelial cells in EB outgrowths at day 5+14. Scale bar, 25 μm. d, vWF-positive endothelial cells (red) at day 5+17. Scale bar, 25 μm. e, Smooth muscle α-actin-positive cells (red) of tube-like structure in EB outgrowths at day 5+14. Scale bar, 100 μm.

FIG. 15. a-c, Neuronal differentiation of murine maGSCs. TH-positive dopaminergic neurons (b) appeared among neurofilament protein M (NFM)-positive neurons (a) on day 12 after plating of EBs at day 5 (day 5+12). c, Overlay of a and b.

d-f, Epithelial cell/hepatocyte differentiation. d, Pan-cytokeratin-positive epithelial cells on day 14 after plating of EBs at day 5 (day 5+14). e, Cytokeratin 18-positive large epithelioid cells on day 5+17. f, Cytokeratin 7-positive bile duct cells on day 5+14. Scale bar, 25 μm (a-f). Nuclear staining with DAPI.

FIG. 16. Lineage-selective differentiation of maGSCs into neural cells. a, b, Uniform expression of nestin (a) and reduced expression of Sox2 (b) in serum-free adherent monoculture of neural precursor cells differentiated from mouse maGSCs. c-f, Differentiation of neural precursor cells into mature neurons positive for type III β-tubulin (TuJ1; c, green) and, SNAP25-positive (d, red) neurons as well as GFAP-positive astrocytes (e) and O1-positive oligodentrocytes (f).

FIG. 17. a-c, Serotonin-positive (a, red) neurons appeared among TuJ1-positive neurons (b). c, Overlay of a and b. d-f, GAD65/67 (glutamic acid decarboxylase)-positive (d, red) GABAergic neurons appeared among TuJ1-positive neurons (e). f, Overlay of d and e.g-i, EAAC1-positive (g, red) neurons appeared among SNAP25-positive neurons (h, green). i, Overlay of g and h. j-l, TH-positive dopaminergic neurons (j) appeared among TuJ1-positive neurons (k). l, Overlay of j and k. Scale bar, 50 μm (a-1).

FIG. 18. a-c, High number of TH-positive dopaminergic neurons (b) appeared among TuJ1-positive neurons (a). c, Overlay of a and b. Scale bar, 50 μm.

FIG. 19. Schematic comparison of the protocols used to differentiate maGSCs into pancreatic cells. a, Protocol I adapted from Blyszczuk et al. includes initial spontaneous differentiation followed by induction of differentiation into the pancreatic lineage. b, Protocol II adapted from Lumelsky et al. begins with selection for nestin-positive cells which are expanded in a mitogenic medium. In contrast to the original protocol bFGF is already withdrawn after three-day application. For induction of pancreatic differentiation, both protocols used the same differentiation medium (N2 medium supplemented with NA).

FIG. 20. RT-PCR analysis of genes important for pancreatic development during EB differentiation according to protocol I. Genes encoding transcription factors important for pancreatic development (Sox17, Pdx1, Shh, Hes1, Ngn3, NeuroD, Isl-1, Nkx2.2, Nkx6.1) and genes encoding functional pancreatic proteins (PC1, insulin 1, insulin2, IAPP and STT) were expressed in a developmentally controlled pattern. Analyses were done at the differentiation stages indicated at the top. Fragment sizes in bp are indicated to the right. GAPDH were used as internal standards.

FIG. 21. RT-PCR analysis of genes important for pancreatic development during EB differentiation according to protocol II. Genes encoding transcription factors important for pancreatic development (Sox17, Pdx1, Shh, Hes1, Ngn3, NeuroD, Isl-1, Nkx2.2, Nkx6.1) and genes encoding functional pancreatic proteins (PC1, insulin 1, insulin2, IAPP and STT) were expressed in a developmentally controlled pattern. Analyses were done at the differentiation stages indicated at the top. Fragment sizes in bp are indicated to the right. GAPDH were used as internal standards.

FIG. 22. Nestin and Pdx1 expression in maGSC-derived cells at early and late stages. Shown are cells cultured according to protocol I at day 5+12 (a) and 5+28 (b) and cells cultured according to protocol II at day 4+7 (c) and 4+19 (d). Double staining was done for nestin (red) and Pdx1 (green). Nuclei were labelled blue (c). Scale bars, 50 μm.

FIG. 23. Nestin and C-peptide expression in maGSC-derived cells at early and late stages. Shown are cells cultured according to protocol I at day 5+12 (a) and 5+20 (b) and cells cultured according to protocol II at day 4+7 (c) and 4+13 (d). Double staining was done for nestin (red) and C-peptide (green). Scale bars, 50 μm.

FIG. 24. Immunofluorescence analysis of maGSCs-derived cells at late stage (5+28). a-c, Shown are cells stained positive for insulin (a, red), C-peptide (b, green); c, overlay of a and b without nuclear staining. d-f, Shown are cells stained positive for glucagons (d, green), somatostatin (e, red), pancreatic polypeptide (f, red) separately. Arrows indicate positive cells. Nuclei are stained blue. Scale bars, 100 μm.

FIG. 25. a, Morphology of 3-(left) and 7-(right) day-old EBs derived from non-human primate SSCs using mass culture methods. Scale bar, 100 μm. b, Morphology of 3-(left) and 7-(right) day-old EBs derived from human SSCs using mass culture methods. Scale bar, 100 μm.

FIG. 26. a-c, Teratomas from maGSCs. The tumors contained abundant differentiation of advanced derivatives of all three embryonic germ layers. Shown are epithelium with intestinal differentiation (a), striated muscle, cartilage (b), and neural tissue (c). Scale bar, 50 μm.

FIG. 27. a, Chimaerism detection by LacZ staining in animals generated from blastocysts microinjected with GFP+SSCs. Images from LacZ-stained various organs from a wildtype mouse (WT), a mouse with high grade of chimerism (H) and a mouse with low grade of chimerisms (L).

b, Genomic DNA isolated from different tissues of animal number 9, 19 and 5 were subjected to genomic PCR using LacZ-specific primers, revealing participation of SSC-derived cells in different tissues. 1, muscle; 2, lung; 3, kidney; 4, spleen; 5, intestine; 6, heart; 7, stomach; 8, testis; 9, liver; 10, brain. +: DNA from ROSA26 mice as positive control; −: DNA from wildtype mice as negative control.

### EXEMPLIFYING SECTION

The following examples are meant to further illustrate, but not limit, the invention. The examples comprise technical features and it will be appreciated that the invention relates also to combinations of the technical features presented in this exemplifying section.

### 1. Isolation of Spermatogonial Stem Cells (SSCs)

### 1.1. Isolation of Murine Spermatogonial Stem Cells

For selection and enrichment of mouse SSCs, a spermatogonia-specific marker Stra8, which is expressed restrictedly to the male germ cells from E14.5 to spermatogonia (Oulad-Abdelghani, M. et al., J Cell Biol 135, 1996, 469-77), was used. The activity of its regulatory sequences enables the enrichment of germline stem cells in transgenic mice (Nayemia, K. et al., Hum Mol Genet 13, 2004, 1451-60). Mice from the transgenic mouse line Stra8-EGFP (FVB) (Nayemia, K. et al., Hum Mol Genet. 13, 2004, 1451-60) were crossed to Rosa26 mice (C57BL6/129Sv; The Jackson Laboratory) to generate the double transgenic mouse line Stra8-EGFP/Rosa26, which has a mixed FVB/C57BL6/129Sv genetic background and was used through the study. Stra8-EGFP8 and Stra8-EGFP/Rosa26 transgenic mouse lines showed specific Stra8-EGFP expression in spermatogonia of adult animals.

For isolation of spermatogonial stem cells (SSCs), testes of adult transgenic males (n=15 mice, 6-week-old) of mouse line Stra8-EGFP/ROSA26 were collected aseptically in serum-free Dulbecco's modified Eagle's medium (DMEM, Gibco/BRL). After decapsulation of the testes, interstitial cells were removed by mechanical agitation and washing with the same medium. The isolated seminiferous tubules were treated with collagenase and trypsin (Boehringer) as described previously (Nayemia, K., et al., Methods Mol Biol 253, 2004, 105-20). Testicular cells (2-3×10⁷ per mouse) were cultured on 0.1% gelatine-coated culture dishes in medium containing 20% fetal calf serum (FCS, selected batch for mouse embryonic stem cell culture; Invitrogen), 1×non-essential amino acids (NEAA; Invitrogen), 2 mM L-glutamine (Invitrogen), 50 μM β-mercaptoethanol (β-ME; Promega), 1× Pen/Strep (PAN) and 4 ng/ml glial-derived neurotrophic factors (GDNF; Tebu) for 4-7 days. Instead of traditional DMEM, KnockOut™ DMEM (Invitrogen) can be used. At the same time, instead of FCS, KnockOut™ serum replacement can be used.

GFP positive cells (1-2×10⁶) were separated by fluorescence-activated cell sorting (FACS) using FACS Vantage cell sorter (Becton Dickinson) equipped with 488-nm argon and 633-nm He—Ne lasers. In all sorting procedures, an initial gating was performed in order to exclude cell debris and cell doublets based on forward- and side-scatter information, and the system was first calibrated with wildtype tissue in order to determine the background signal for GFP fluorescence. Green fluorescence was measured through a 530-nm/30-nm bandpass filter after illumination with the 488-nm line of an argon ion laser. The sorted GFP+ cells were pelleted and cultured on 0.1% gelatine-coated culture dishes in medium containing 15% FCS, 1×NEAA, 2 mM L-glutamine, 50 μM β-ME and 4 ng/ml GDNF for 1 week.

Transplantation of Murine SSCs into Seminiferous Tubules of Testes

At 6-8 weeks of age, the recipient mice were injected i.p. with busulfan (40 mg/kg body weight), which destroys endogenous spermatogenesis. Recipients were used for transplantation at 4 weeks after injection. To prove stem cell activity of FACS-sorted GFP+-cells, approximately 1-3×10⁵ FACS-sorted GFP+ cells were transplanted via rete testis into seminiferous tubules of one testis (Nayernia, K., et al., Methods Mol Biol 253, 2004, 105-20). As external control, busulfan treated non-transplanted males were used. After 20 weeks, recipient mice were killed and testes were examined by LacZ staining as described (Patapoutian, A., et al., Development 118, 1993, 61-9).

After 5-6 months, regeneration of normal spermatogenesis was observed by LacZ+ cells (data not shown). These results indicate that GFP+ cells fulfil the criteria of SSCs and are not derived from testicular teratomas. Testicular teratomas are rare in most inbred mouse strains and occur spontaneously at a rate of 1-5% in 129/Sv inbred strains (Stevens, L. C. & Hummel, K. P., J Natl Cancer Inst 18, 1957, 719-47). In a mixed C57BL/6 and 129/Sv genetic background, a lower number of tumour suppressor gene TRP53-deficient mice develop testicular tumours than on a pure 129/Sv genetic background (Donehower, L. A. et al., Mol Carcinog 14, 1995, 16-22). We used the transgenic mouse line which has a mixed FVB/C57BL6/129Sv genetic background. We did not observe testicular tumours out of 15 males in this mixed genetic background.

SSCs from adult mice with three different genotypes (C57BL/6, 129/Ola and FVB) were also isolated without the genetic selection. The testicular cells were first cultured in DMEM containing 20% FCS, 1×NEAA, 2 mM L-glutamine, 50 μM β-ME, 1× Pen/Strep and 4 ng/ml GDNF on 0.1% gelatine-coated culture dishes at 37° C. and 5% CO₂ overnight. After removal of attached cells, spermatogonia, which remained in suspension, were collected and cultured on fresh gelatine-coated tissue culture dishes in the same medium for 1-2 weeks.

1.2. Isolation of Spermatogonial Stem Cells from Non-Human Primate and Human

For isolation of spermatogonial stem cells from adult non-human primate, approximately 20 g of testicular tissue was used for each cell isolation procedure. After decapsulation, the testes were first washed in DPBS, minced into small pieces and suspended in DMEM supplemented with penicillin and spreptomycin.

For isolation of human SSCs, testis biopsies obtained from patients (adult male) was first washed in Dulbecco's phosphate buffered salt solution (DPBS) and then minced into small pieces and suspended in DMEM supplemented with penicillin and spreptomycin.

The testicular tissues were then treated with one of the two following methods.

Method I: The minced testicular tissues were incubated in Hanks' Balanced Salt Solution (HBSS) containing 1 mg/ml collagenase for 15-30 min at 37° C. After centrifugation for 5 min at 1000 rpm, the pellets were washed three times in pure HBSS medium, and resuspended in 5 ml Trypsin/EDTA (1×Trypsin/EDTA, 0.05%/0.02% solved in DPBS). After repeated pipetting, 10-20% vol. FCS was added. The large tissue residues were removed with sterile tips, and small residues of tissue were removed by filtrating through 70 μm nylon filters (BD Falcon Cell Strainer).

Method II: Minced pieces were incubated in DMEM containing 1 mg/ml collagenase, 1 mg/ml hyaluronidase, 1 mg/ml trypsin and 5 μg/ml DNase at 32° C. for 60 min in a shaking water bath. After three washes in DMEM and removal of most of interstitial cells, seminiferous tubular fragments were incubated in DMEM containing 1 mg/ml collagenase, 1 mg/ml hyaluronidase, and 5 μg/ml DNase at 32° C. for 45 min. After repeated pipetting, tubular fragments were centrifuged at 30×g for 2 min. Cells in the supernatant were collected, and filtered through 70 μm nylon filters.

The flow through was centrifuged for 5 min at 1000 rpm. The resulting pellet was resuspended in DMEM/F12 medium (Invitrogen) containing 20% FCS, 1×NEAA, 2 mM L-glutamine, 50 μM β-ME, 1× Pen/Strep, 4 ng/ml bFGF (Tebu) and 4 ng/ml GDNF, and incubated on 0.1% gelatine-coated culture dishes at 37° C. and 5% CO₂ overnight. After removal of attached cells, spermatogonia, which remained in suspension, were collected and cultured on fresh gelatine-coated tissue culture dishes in the same medium for 3-10 days.

### 2. Culture of Spermatogonial Stem Cells (SSCs)

### 2.1. Culture of Murine Spermatogonial Stem Cells

The Stra8-EGFP+ cells that had been tested for the contribution of spermatogenesis were used to establish the conditions required to convert these cells into pluripotent stem cells. To maintain the GFP+ cells at undifferentiated state, four different culture conditions were tested. Condition I: Cells were cultured in gelatine-coated culture dishes with basic medium, DMEM (high glucose formulation; Gibco/BRL) supplemented with 15% FCS, 2 mM L-glutamine, 50 μM β-ME, 1×NEAA. Condition II: Cells were cultured in gelatine-coated culture dishes with basic medium containing 10³ units/ml leukaemia inhibitory factors (LIF; ESGRO, Chemicon). Condition III: Cells were cultured on a feeder layer of mitomycin C-inactivated mouse embryonic fibroblasts with basic medium. Condition IV: Cells were cultured on a feeder layer with basic medium containing 10³ units/ml LIF (=embryonic stem cell culture condition). Standard embryonic stem cell (ESC) culture condition as described (Guan, K., et al., Cytotechnology 30, 1999, 211-26) was used for cultivation of mouse MPI-II ESCs isolated from inner cell mass of the blastocysts (Voss, A. K., et al., Exp Cell Res 230, 1997, 45-9). Instead of traditional DMEM, KnockOut™ DMEM (Invitrogen) can be used. At the same time, instead of FCS, KnockOut™ serum replacement can be used.

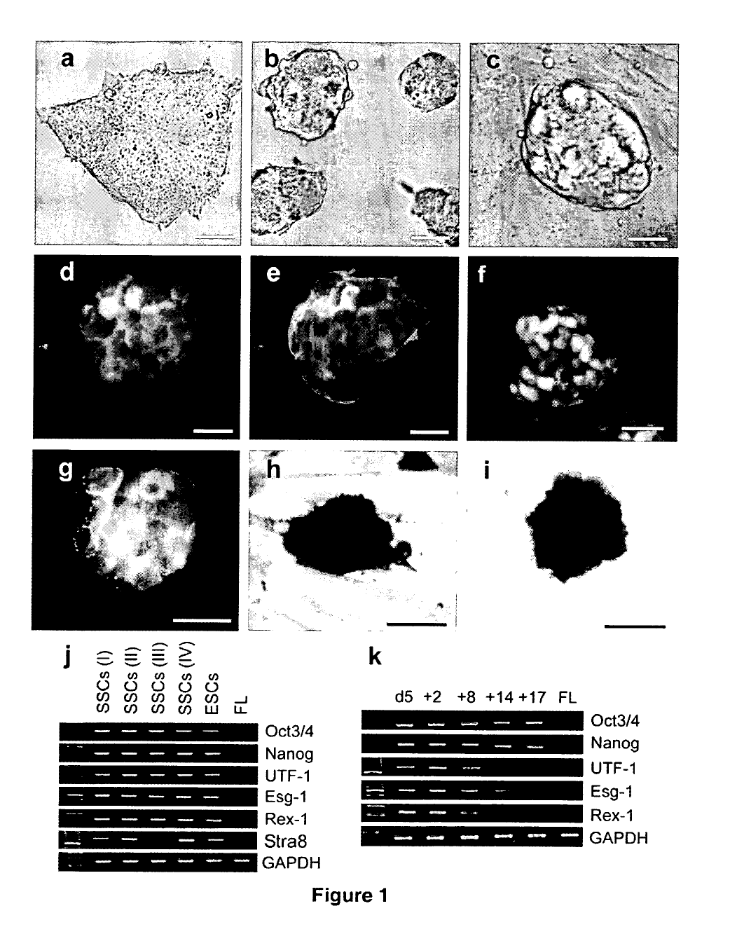

The GFP+SSCs were first propagated in basic medium (condition I) containing 4 ng/ml GDNF for 2 weeks. The cells were dissociated by trysin/EDTA solution (0.2% Trysin/0.02% EDTA=1:1 in PBS) and passaged at a dilution of 1:3 every 4-5 days. Afterwards, the cells were cultured in four different conditions. When the GFP+SSCs were cultured under condition I, the majority of the colonies that formed were similar to cultured epiblast cells (FIG. 1a). However, when the cells cultivated in the medium containing leukaemia inhibitory factor (LIF; condition II) or on a feeder layer of mitomycin C-treated mouse embryonic fibroblasts (condition III), tightly packed embryonic stem cell (ESC)-like colonies appeared after 2-3 passages (FIG. 1b). These ESC-like colonies can be expanded with standard ESC culture conditions (both feeder layer and LIF applied; condition IV) at a dilution of 1:3 to 1:6 every day. We have successfully established four cell lines (SSC5, SSC6, SSC10 and SSC15) out of fifteen mice (27%). This is much higher than the rate of spontaneous teratoma formation in strain 129 (1-5%), indicating that the GFP+ cells are unlikely to be derived from testicular teratomas. Two cell lines, SSC5 and SSC6, have now been passaged for more than 30 passages (FIG. 1c). The morphology of the tightly packed ESC-like colonies did not change as long as the cells were kept in condition IV. To prove that a single cell gives rise to continuous growing cultures, for each cell line, we generated at least six sub-lines derived from single cells. Based on the experience with isolation of SSCs from the GFP+ transgenic mice, it was possible to identify the cells morphologically, allowing to rule out that the isolation is restricted to the transgenic mice. ES-like cell lines from mice with three different genotypes (C57BL/6, 129/Ola and FVB) were successfully derived without the genetic selection. Briefly, after cultivation in DMEM containing 20% FCS, 1×NEAA, 2 mM L-glutamine, 50 μM β-ME, 1× Pen/Strep and 4 ng/ml GDNF for 1-2 weeks, the testicular cells were then cultured under four different conditions and passaged every 3-4 days. ESC-like colonies appeared after 2-3 passages under conditions II to IV. To eliminate other cell types, two methods were used: a) Pre-plating of the isolated testicular cells was performed at each passage. b) ESC-like colonies were mechanically isolated and enriched for another 2-3 passages.

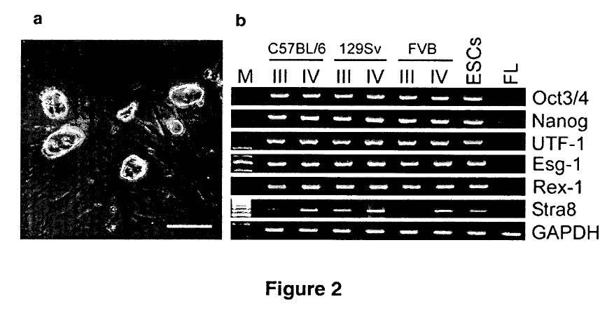

Pre-Plating: briefly, the testicular cells were harvested from the tissue culture dishes by treatment with 0.1% trypsin and 0.01% EDTA in phosphate-buffered saline (PBS). The dissociated cells were replated in 0.1% gelatine-coated culture dishes and put back to the incubator. The Sertoli, fibroblasts and endothelial cells attached to the culture dishes after about 1.5 hours. The floating cells were collected and transferred to a fresh 0.1% gelatine-coated tissue culture dish. ES-like cells (FIG. 2a) appeared in two of eleven C57BL/6 (18%), in one of seven FVB (14%) and in one of three 129/Ola (33%) mice.

### 2.2. Culture of Non-Human Primate and Human Spermatogonial Stem Cells

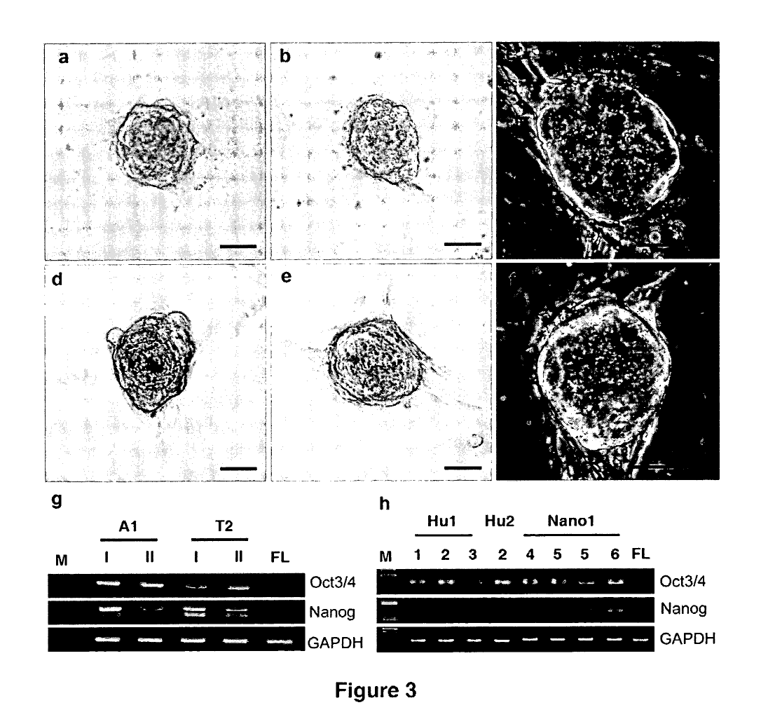

Mouse SSC cultures indicated that both feeder layer and LIF are preferable for the establishment of ESC-like cells. Therefore, culture conditions II to IV were applied, which had been used for the cultivation of mouse ESC-like cells derived from SSCs, for the establishment of human and non-human primate ESC-like cells. Human as well as non-human primate SSCs were dissociated by trypsin/EDTA solution (0.2% Trypsin/0.02% EDTA=1:1 in PBS) and passaged at a dilution of 1:3 every 5-7 days. ESC-like colonies derived from non-human primate SSCs appeared after 2-3 passages (ca. two-week culture; FIGS. 3a, b), but with low efficiency. However, no ESC-like colonies developed from human SSCs during a two-month culture. This indicates that human as well as non-human primate SSCs might differ from mouse SSCs.

To optimize the culture condition, DMEM/F12 (1:1; Invitrogen) was applied instead of DMEM as basic medium and several growth factors for long-term culture were added: GDNF (4-50 ng/ml), bFGF (1-10 ng/ml), LIF (10³ Units/ml), BIO (Calbiochem) and 1×B27 (Invitrogen). Collagenase IV (1 mg/ml in PBS) was used instead of trypsin/EDTA for passaging the cells. First, it was found that DMEM/F12 instead of DMEM as basic medium resulted in better proliferation of human as well as non-human primate SSCs. Tightly packed ESC-like colonies derived from human SSCs after 7 passages (about 1 month; FIG. 3d, e) when cells are cultured in DMEM/F12 medium containing 15% FCS, 2 mM glutamine, 1×NEAA, 50 μM β-ME, 10 ng/ml GDNF, 2 ng/ml bFGF, 10³ Units/ml LIF and 1×B27. Specially, application of BIO (1 μM) optimized the formation of ESC-like colonies both in non-human (FIG. 3c) and in human (FIG. 3f) cultures.

### 3. Phenotypic Characterization of Cultured SSCs and ESC-Like Cells

### 3.1. Phenotypic Characterization of Cultured Murine SSCs and ESC-Like Cells

For characterization by flow cytometry, the SSCs as well as ESCs were harvested from the tissue culture dishes by treatment with 0.1% trypsin and 0.01% EDTA in phosphate-buffered saline (PBS). To eliminate the feeder cells, pre-plating method was applied. Briefly, the harvested cells were replated in 0.1% gelatine-coated culture dishes and put back to the incubator. The feeder cells attached to the culture dishes after about 1.5 hours. The floating cells were collected and fixed in 4% paraformaldehyde. The fixed cells were washed twice with two volumes of PBS and then blocked with 1% BSA at RT for 1 hour. The cells, in solution at a concentration of 0.5×10⁶ cells/ml, were stained for 30 min with an empirically determined amount of each antibody. Labelled cells were thoroughly washed with two volumes of PBS. The following primary antibodies were used: mouse anti-SSEA-1 (clone MC480, Developmental Studies Hybridoma Bank, DSHB), mouse anti-SSEA-3 (clone MC631, DSHB), mouse anti-SSEA-4 (clone MC893-70, DSHB), mouse anti-Oct4 (clone 9E3.2, Chemicon), phycoerythrin (PE)-conjugated anti-mouse c-kit, Thy1, Ter119, Sca-1 and CD34 (Becton Dickinson). Cy3-conjugated goat anti-mouse IgG+IgM was used as secondary antibodies. The labelled cells were analyzed on a FACScan (Becton-Dickinson) by collecting 50,000 events with the Cell Quest software program (Becton-Dickinson).

For characterization by immunocytochemistry, cultured SSCs were subjected to specific immunostaining for SSEA-1 (mouse monoclonal antibody, clone MC480, Developmental Hybridoma Bank), Oct4 (mouse monoclonal antibody, clone 9E3.2, Chemicon), SSEA-3 (mouse monoclonal antibody, clone MC631, Developmental Hybridoma Bank), and GFP (rabbit polyclonal antibody, abcam) using the procedure as described (Wobus, A. M. et al., Methods Mol Biol 185, 2002, 127-56).

Characterization of cultured SSCs by ALP staining was carried out using a Sigma alkaline phosphatase substrate kit (86C-1 KIT) according to manufacturer's protocol.

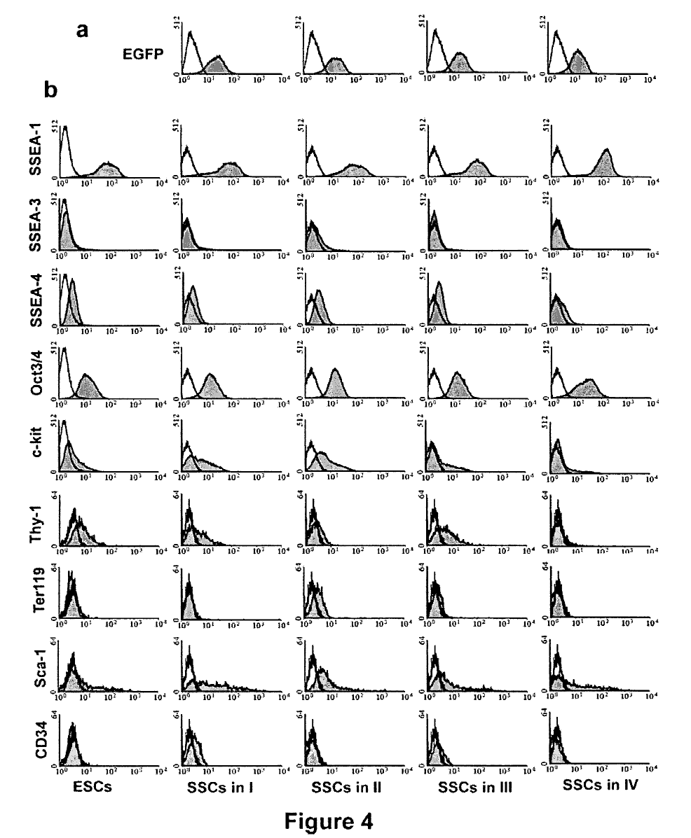

Results: The phenotype of cultured GFP+SSCs is dependent on different culture conditions (FIGS. 1, 4). Similar to mouse ESCs, GFP+SSCs cultured in all four conditions (I-IV) were strongly positive for the cell surface marker stage-specific embryonic antigen-1 (SSEA-1; FIG. 1e; FIG. 4) and the germline-specific transcription factor Oct3/4 (FIG. 1f; FIG. 3) that characterize undifferentiated mouse ESCs, weakly positive for SSEA-4 and Sca-1, and completely negative for SSEA-3, Ter119 and CD34 (FIG. 4). In common with ESCs, the SSCs cultured with condition III or IV didn't express SSEA-3 (Damjanov, I. et al., Am J Pathol 108, 1982, 225-30), but cells cultured with conditions I or II stained partially for SSEA-3 (FIG. 1g; FIG. 4). The c-kit receptor is expressed in differentiating spermatogonia, but not in SSCs (Rossi, P., et al., J Endocrinol Invest 23, 2000, 609-15). We found a higher number of cells positive for c-kit when cultured without feeder layers (condition I: 26.1% and II: 27.2%) than when cultured on feeder layers (condition III: 15.9% and IV: 8.6%) (FIG. 4). Thy-1 is believed as a surface marker of SSCs in mouse testis (Kubota, H., et al., Biol Reprod 71, 2004, 722-31), and can be also detected on ESCs (Ling, V. & Neben, S., J Cell Physiol 171, 1997, 104-15). However, GFP+ cells cultured in the presence of LIF (conditions II and IV) are lower positive or are negative for Thy-1 than those cultured without LIF (conditions I and III) (FIG. 4). Alkaline phosphatase (ALP) is highly expressed in ESCs and PGCs, but not in SSCs (Kanatsu-Shinohara, M. et al., Cell 119, 2004, 1001-12). In our study, ALP was strongly expressed in the GFP+ cells cultured in condition IV (FIG. 1h) whereas a “mixed colony phenotype” was revealed in conditions I to III (FIG. 1i). These results suggest that mouse SSCs respond to the culture conditions in line with the previous studies (Nagano, M., et al., Biol Reprod 68, 2003, 2207-14; Kubota, H., et al., Proc Natl Acad Sci USA 101, 2004, 16489-94). Under the standard ESC culture condition, they can acquire ESC properties. Therefore, we name these cultured GFP+ES-like cells multipotent adult germline stem cells (maGSCs) in order to differ from the SSCs.

### 3.2. Phenotypic Characterization of Cultured Non-Human Primate and Human SSCs

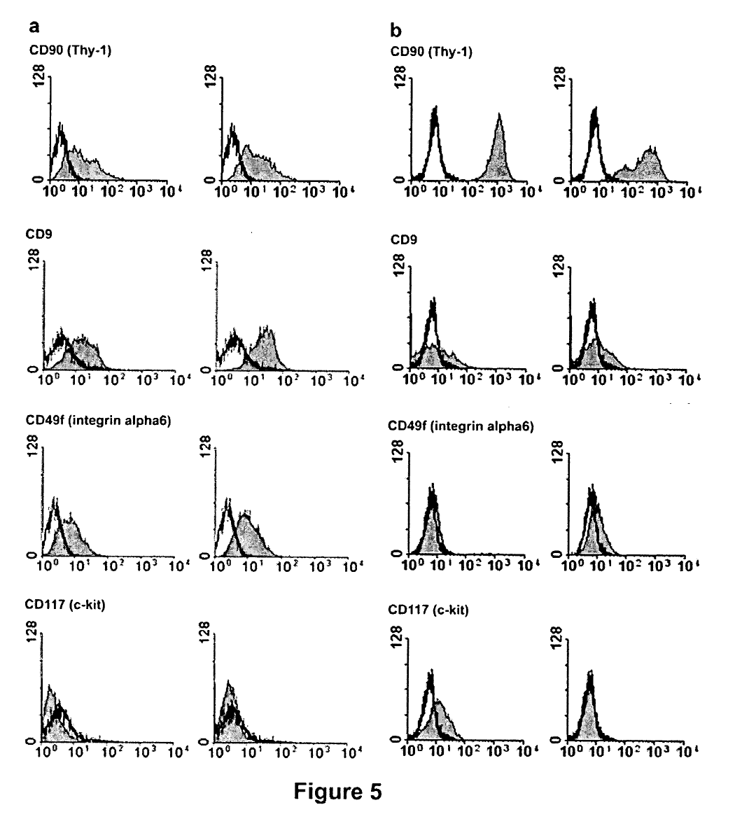

For characterization by flow cytometry, the cultured non-human primate and human were harvested from the tissue culture dishes by treatment with 0.1% trypsin and 0.01% EDTA in phosphate-buffered saline (PBS). The dissociated cells, in solution at a concentration of 0.5×10⁶ cells per ml, were stained for 30 min with an empirically determined amount of each antibody. Labelled cells were thoroughly washed with two volumes of PBS. The following primary antibodies were used: phycoerythrin (PE)-conjugated anti-human CD9, and CD117 (c-kit), and FITC-conjugated anti-human CD90 (Thy1) and CD49f (Integrin α6) (Becton Dickinson). PE-IgG1 and FITC-IgG1 was used as negative control. The labelled cells were analyzed on a FACScan (Becton-Dickinson) by collecting 50,000 events with the Cell Quest software program (Becton-Dickinson). Non-human primate SSCs cultured in DMEM/F12 medium containing 15% FCS, 2 mM glutamine, 1×NEAA and 50 μM β-ME are partially positive for CD90 (Thy-1), CD9 and CD49f (integrin α6), but negative for CD117 (c-kit) (FIG. 5a, left panel). When non-human primate SSCs cultured in medium containing additional GDNF (10-50 ng/ml), bFGF (1-10 ng/ml), LIF (10³ Units/ml) and 1×B27, more cells are positive for CD90, CD9 and CD49f (FIG. 5a, right panel). Human SSCs cultured in DMEM/F12 medium containing 15% FCS, 2 mM glutamine, 1×NEAA and 50 μM β-ME are strong positive for CD90, very low positive or negative for CD9, CD49f and CD117 (c-kit) (FIG. 5b, left panel). When human SSCs cultured in medium containing additional GDNF (10-50 ng/ml), bFGF (1-10 ng/ml), LIF (10³ Units/ml) and 1×B27, almost all cells are negative for CD117 (FIG. 5b, right panel). Little more cells are positive for CD49f.

### 4. RT-PCR Analysis of Cultured SSCs and ESC-Like Cells

### 4.1. RT-PCR Analysis of Cultured Murine SSCs and ESC-Like Cells

Mouse SSCs cultured with different conditions (I-IV) and ESC-like cells were collected for total RNA isolation (Promega). 150 ng of DNase-treated RNA was used for first-strand cDNA synthesis. One-tenth of the cDNA reaction was taken as PCR template and amplified for 25-35 cycles. GAPDH was used as an internal control. Stem cell-specific maker genes were analysed.

Results: The RT-PCR analysis showed that the cultured cells under all four conditions, like ESCs, expressed genes encoding transcription factors Oct3/4, nanog, UTF-1, Esg-1 and REX-1 (FIG. 1j) which are important in maintaining undifferentiated ESCs. Surprisingly, cells cultured with LIF (conditions II and IV) expressed higher level of Stra8 than those cultured without LIF (conditions I and III), suggesting that GFP+ cells are susceptible to LIF.

The cultures derived from wildtype mice of three different strains (C57BL6, FVB and 129/Ola), similar to GFP+ cells, express Stra8, Oct3/4, nanog, UTF-1, Esg-1 and REX-1 and are susceptible to LIF (FIG. 2).

### 4.2. RT-PCR Analysis of Cultured Non-Human Primate and Human SSCs

Non-human primate and human SSCs cultured with different conditions were collected for total RNA isolation (Promega). 150 ng of DNase-treated RNA was used for first-strand cDNA synthesis. One-tenth of the cDNA reaction was taken as PCR template and amplified for 25-35 cycles. GAPDH was used as an internal control. Stem cell-specific maker genes were analysed.

Results: The RT-PCR analysis showed that the cultured cells under all four conditions, like ESCs, expressed genes encoding transcription factors Oct3/4, nanog (FIG. 3g) which are important in maintaining undifferentiated ESCs.

5. In Vitro Differentiation and Characterization of maGSCs
5.1. In Vitro Differentiation and Characterization of Murine maGSCs

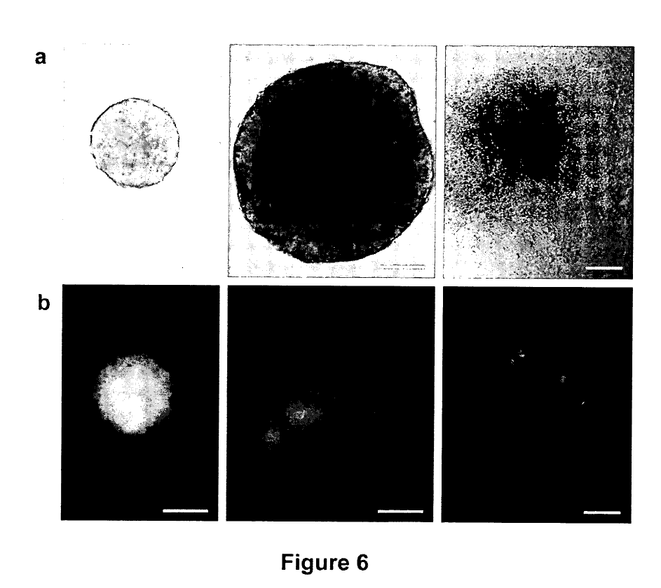

For differentiation into ectodermal, mesodermal and endodermal lineages, mouse maGSCs were cultivated as embryoid bodies (EBs) (FIG. 6a) in hanging drops in Iscove's modified Dulbecco's medium (IMDM, Gibco) supplemented with 20% FCS, 2 mM L-glutamine, 1×NEAA and 50 M β-ME as described for standard mouse ESC differentiation (Guan, K., et al., Cytotechnology 30, 1999, 211-26). Briefly, maGSCs were cultured without LIF for 2 passages and the feeder cells were eliminated by pre-plating method before cultivation as EBs. Cells (n=400) in 20 μl of differentiation medium were placed on the lids of petri dishes filled with phosphate-buffered saline (PBS) and incubated in hanging drops for 2 days and in bacteriological petri dishes for 3 days. At day 5, single EBs were transferred into each well of gelatine-coated 24-well plates for morphological analyses, into 6-cm tissue culture dishes containing 4 cover slips (25-35 EBs per dish) for immunocytochemistry (see below), or into 6-cm tissue culture dishes for RT-PCR analyses (see below). The differentiation of maGSCs was compared to those of mouse MPI-II ESCs, which were differentiated using the same method described above.

Results: An overall decrease in GFP+ cell populations was observed upon EB differentiation (FIG. 6b). In parallel, the expression of Oct3/4, nanog, UTF-1, Esg-1 and REX-1 was gradually decreased during differentiation (FIG. 1k). This is similar to the ESC differentiation under the same condition (data not shown). To determine whether maGSCs can spontaneously differentiate into derivatives of three primary germ layers, the expression of a panel of cell-specific genes and proteins during EB differentiation was examined. It was found that the cells expressed the analysed genes and proteins in a developmentally controlled manner.

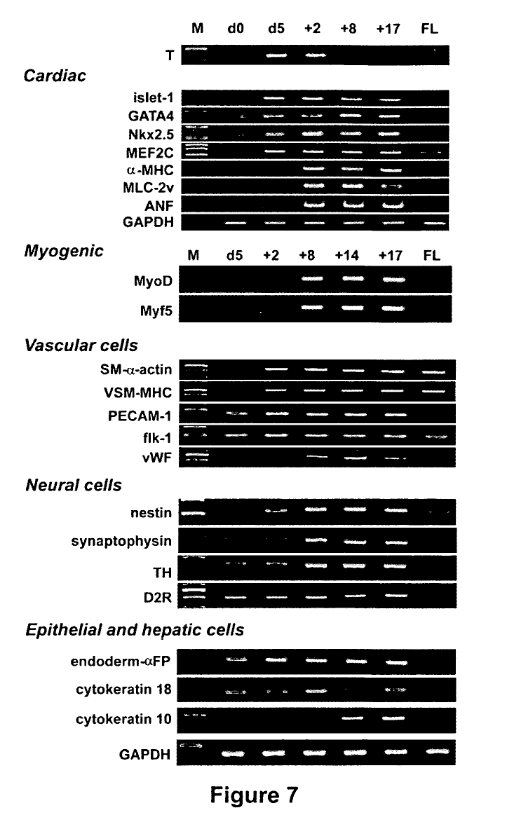

Differentiation of mesodermal lineages (for example, cardiac, skeletal muscle, and vascular cells) was confirmed by the expression of the early mesoderm marker Brachyury (T) gene (King, T., et al., Mech Dev 79, 1998, 29-37) as well as lineage-specific genes and proteins. The expression of T gene was maximal at early differentiation stages (FIG. 7). The mRNAs encoding cardiac transcription factors islet-1 (Laugwitz, K. L. et al., Nature 433, 2005, 647-53), GATA4, Nkx2.5 and MEF2C appeared at a high level already at day 5 whereas high levels of mRNAs encoding cardiac-specific genes, such as α-myosin heavy chain (α-MHC), ventricular isotype 2 of myosin light chain (MLC2v) and atrial natriuretic factor (ANF) appeared two days later (day 5+2; FIG. 7). Spontaneously and rhythmically contracting cells appeared as clusters and were identified in approximately 70% of the individual EBs (n=144; three independent experiments) at day 5+2 and increased to as many as 90% of the EBs at day 5+5 comparable to cardiac differentiation of ESCs (86%).

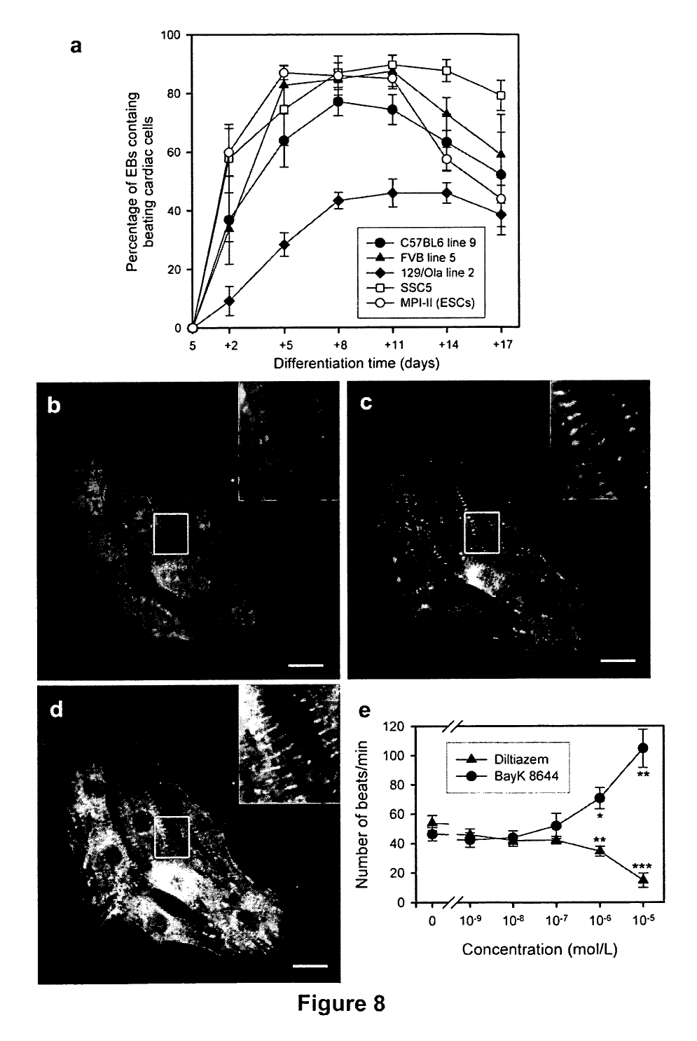

In addition, maGSCs derived from wildtype mice of three different strains (C57BL6, FVB and 129/Ola) can also spontaneously differentiate into cardiac cells by inducing EB formation (FIG. 8). Spontaneously and rhythmically contracting cells appear as clusters and are identified in approximately 40% of the individual EBs (n=144; three independent experiments) derived from line 9 of C57BL6 strain at differentiation day 5+2 (2 days after plating of 5-day-old EBs on gelatine-coated culture dishes), increased to as many as 80% of the EBs by day 5+8, and declined to 50% by day 5+17 due to the overgrowth of other cells (FIG. 8a). The percentages of beating EBs derived from line 9 of C57BL6 strain during differentiation is similar to those derived from line SSC5 of double transgenic mouse and to those derived from ESCs (FIG. 8a). Similar results were seen in FVB line 5 (FIG. 8a). However, lower differentiation efficiency was seen in 129/Ola line 2 in comparison to line SSC5 and ESCs (FIG. 8a). The remainder of the experiments then focused on EBs or cardiac cells derived from C57BL6 line 9 and SSC5. RT-PCR assays show that cardiomyocytes derived from C57BL6 line 9, similar to those derived from SSC5 cell line, express cardiac gene products in a developmentally controlled manner (data not shown).

Cardiomyocyte phenotype of these contracting areas can be demonstrated by immunostaining of proteins relevant for myocyte contraction. For a better demonstration of the structural organization of intracellular, especially sarcomeric proteins, cardiomyocytes are isolated as single cells from the beating areas of EBs (at day 5+4) mechanically using a micro-scalpel under an inverted microscope (Wobus, A. M. et al., Methods Mol Biol 185, 2002, 127-56). Tissues were collected in Tyrode's solution with (in mM) 140 NaCl, 4 KCl, 1 MgCl₂, 10 glucose, 5 HEPES (pH 7.4 with NaOH). The isolated clusters were digested in collagenase type 2 (310 U/ml, Worthington) supplemented Tyrode's solution with 30 μM CaCl₂ at 37° C. for 30 minutes. For the isolation of small cardiac clusters, the incubation time was shortened to 15 minutes. The cells were then resuspended and dissociated in 100 μl of KB medium at 37° C. for 30 minutes. The cell suspensions were transferred into tissue culture plates containing gelatin-coated cover slips and incubated in differentiation medium at 37° C. overnight. Cardiomyocytes began rhythmical contractions after 24 hours and were used for immunostaining 3 days later.

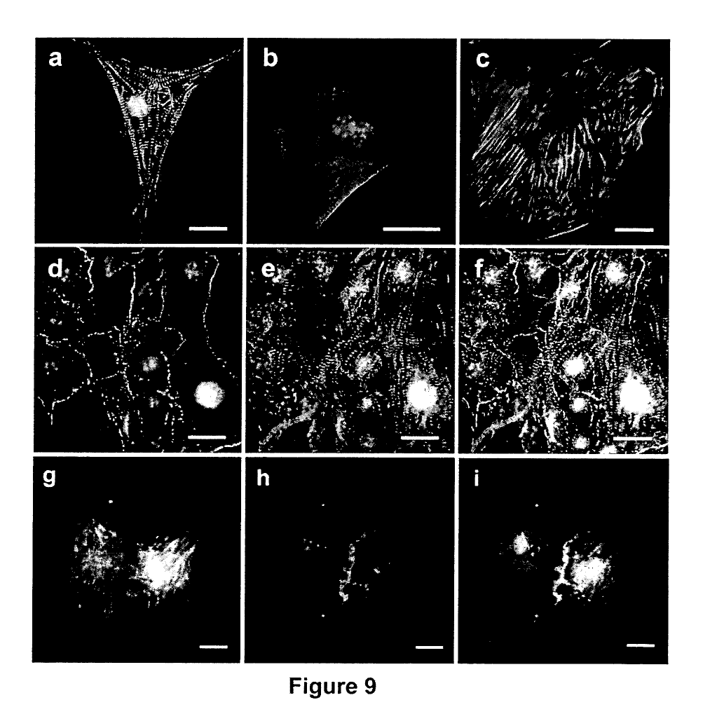

Isolated cardiomyocytes were analysed by specific immunostaining for sarcomeric α-actinin (rabbit polyclonal antibody, clone 653, kindly from Prof. DO Fürst, University of Potsdam, Germany), α-myosin heavy chain (mouse monoclonal antibody, clone MF20, Developmental Hybridoma Bank), cardiac troponin T (Ab-1, mouse monoclonal antibody, clone 13-11, Lab Vision). Accordingly, single cardiomyocytes isolated from beating areas exhibit sarcomeric striations when stained with α-sarcomeric actinin (FIG. 9a), organized in separated bundles. They also stain with sarcomeric MHC and cardiac troponin T in a partially or completely organized pattern (FIG. 9b, c), but don't stain with nebulin, a specific marker for skeletal muscle cells (Furst, D. O., et al., J Cell Biol 109, 1989, 517-27) (data not shown).

In cardiac muscle, where Ca²⁺ influx across the sarcolemma is essential for contraction, the dihydropyridine (DHP)-sensitive L-type calcium channel (DHP receptor) represents the major entry pathway of extracellular Ca²⁺. Using immunofluorecence staining with a specific α1c antibody we could show that α1 subunit of L-type calcium channels are expressed in cardiomyocytes at day 5+6 in a striated pattern (FIG. 8b-d). The function of L-type calcium channels is examined in beating cardiac clusters at day 5+15 by evaluating chronotropic effects of cardioactive drugs. L-type calcium channel activator (S)-BayK 8644 (1,4 dihydropyridine-type) shows dose-dependent positive chronotropic effects on the beating frequency of maGSC-derived cardiomyocytes whereas L-type calcium channel blocker diltiazem (1,5-benzothiazepine-type) shows dose-dependent negative chronotropic effects (FIG. 8e). Treatment with 10⁻⁵ mol/L (S)-BayK 8644 doubles the beating frequency. Treatment with 10⁻⁵ mol/L diltiazem almost completely blockes the contraction. Contractions recover to a normal rate 24 hours after removal of the drug. These results indicate that functional L-type calcium channels exist in the maGSC-derived beating cardiomyocytes.

To analyse the cell-to-cell coupling in the synchronously contracting cardiac clusters, a double-staining in the cells with either pan-cadherin (mouse monoclonal antibody, clone CH-19, Sigma) and α-actintin or connexin 43 (Cx43; monoclonal antibody, clone 4E6.2, Chemicon) and α-actintin was performed. It was found that pan-cadherin localized at cell-cell junctions indicating the presence of adhering junctions between cardiac cells (FIG. 9g-i). In addition, Cx43 staining indicates the presence of gap junctions between cells in cardiac clusters (FIG. 9d-f).

Functional coupling between cells is confirmed by fluorescence recovery after photobleaching (FRAP) analysis. Briefly, cardiac clusters were loaded with the membrane-permeant fluorescent dye calcein AM (5 μmol/L; Invitrogen) in Ca²⁺-free Tyrode solution for 20 min at 37° C. After washing away the excess extracellular fluorescent dye to prevent further loading, the cultures were bathed in Ca²⁺-free Tyrode solution and placed on the stage of a Zeiss LSM 5 PASCAL laser-scanning confocal microscope. Using Zeiss software, a rectangular region encompassing 30-50% of a single cell within a cell cluster was selected and its fluorescence was bleached by a high-intensity laser pulse (488 nm, 3-5 s duration). This caused immediate loss of calcein fluorescence emission recorded through a 505 nm long-pass emission filter. Calcein redistribution from adjacent unbleached cells through connexin pores into the bleached region of interest (ROI) was recorded in subsequent confocal images acquired at 30 s intervals for up to 10 min using a low-intensity laser pulse. Fluorescence recovery within ROI was plotted as a function of time and fit to a single exponential function: I_{ROI} (t)=A [1−exp (−kt)], where I_{ROI} (t) is the ROI fluorescence intensity at time t, A is the amplitude of fluorescence recovery, and k is the rate of recovery. The latter is considered a measure of gap junction permeability. For the inhibitor studies, the cells were incubated in Ca²⁺-free Tyrode solution containing the gap junction uncoupler 3β-hydroxy-11-oxoolean-12-en-30-oic acid 3-hemisuccinate (carbenoxolone, 50 μmol/L; Sigma).

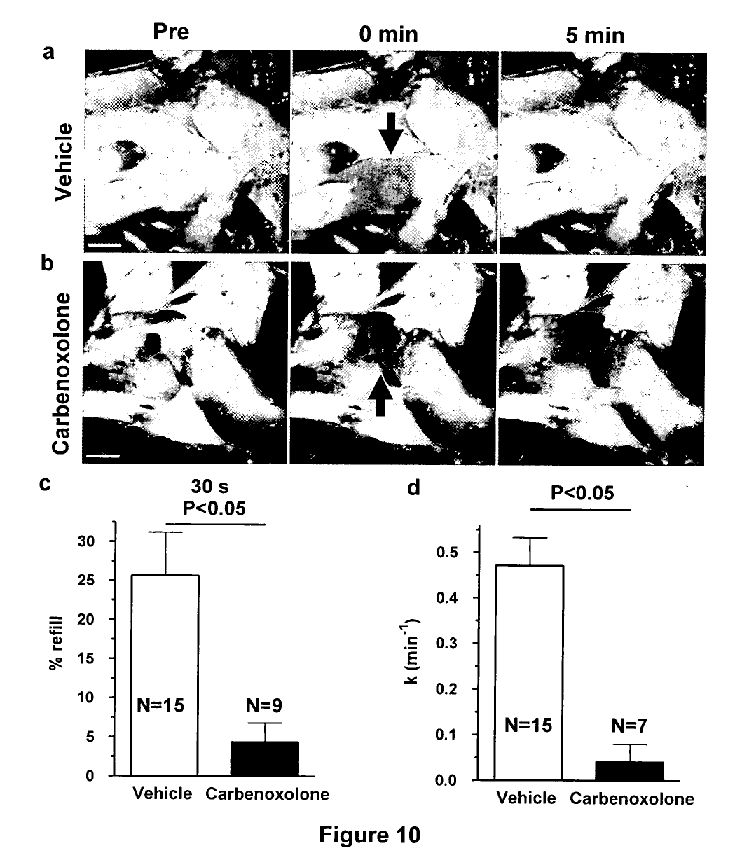

Fluorescence recovery of a gap junction permeable dye (calcein AM) is observed consistently for cardiac cells within 5 minutes after photobleaching (FIG. 10a). When cells are exposed to carbenoxolone (50 μmol/L), a gap junction uncoupler, the fluorescence recovery is disrupted (FIG. 10b). Quantification of the fluorescence intensity verifies that cardiac cells recover to 25.6±5.6% (n=15) after 30 seconds and the gap junction uncoupler carbenoxolone significantly block the percentage of refill to 4.3±2.4% (n=9; P<0.05) (FIG. 10c). The magnitude of functional gap junctions (or the gap junction permeability) is assessed by the rate of fluorescence recovery (k) which reflects the diffusion of calcein AM from unbleached neighbors into a laser-bleached cell. The recovery rate (k) in maGSC-derived cardiomyocytes is 0.47±0.06 min⁻¹ (n=15) and the application of the gap junction uncoupler carbenoxolone results in the significant lower rate of recovery (k=0.04±0.04 min⁻¹; n=7; P<0.05; FIG. 10d). Taken together, these observations suggest that gap junctions function as a conduit of intercellular communication between cardiomyocytes and play an important role in cell-to-cell communication essential for synchronization of myocardial contractile activity and intact electromechanical coupling.

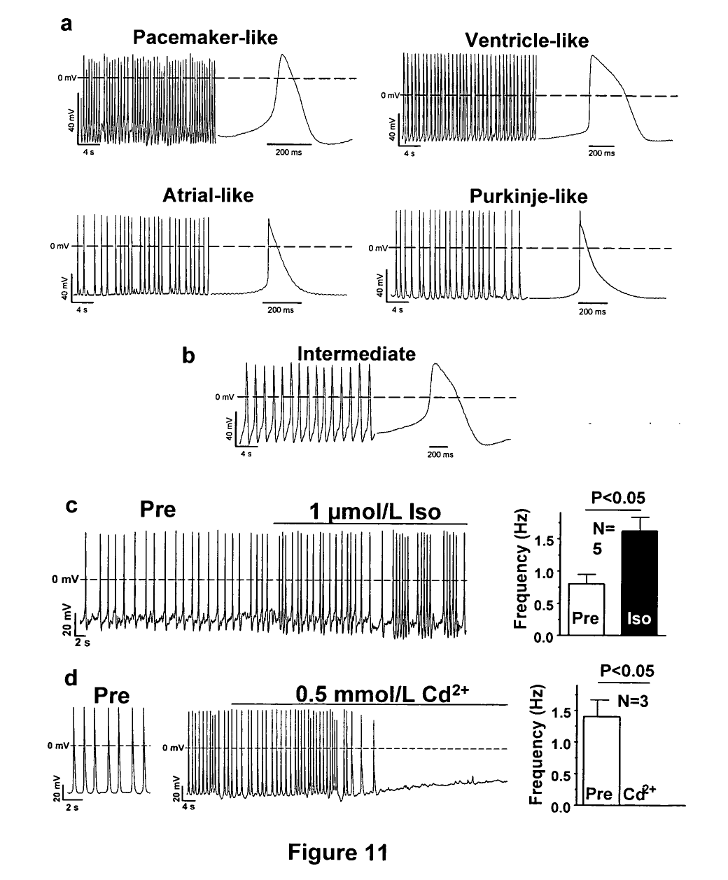

In mature cardiac cells, depolarization of the cell membrane during the action potential activates L-type Ca²⁺-channels leading to Ca²⁺ influx and subsequent release of Ca²⁺ from intracellular calcium stores. To characterize whether mouse SSC-derived cardiomyocytes could enter a fully differentiated cardiac phenotype, the dissociated cardiomyocytes were analysed by patch-clamp electrophysiology. Mouse SSC-derived cardiomyocytes show spontaneous action potentials (APs, FIG. 11a). We examined the shape and properties of APs from 68 single beating cardiomyocytes. Four major types of action potentials characteristic for pacemaker-(n=8), ventricle-(n=23), atrial-(n=9), and Purkinje-like cells (n=9) are found in SSC-derived cardiomyocytes (FIG. 11a; Table 3) with distinct morphologies at a middle differentiation stage (day 5+9) similar to those found in ESC-derived cardiomyocytes (FIG. 11a). This classification was based on the shapes (FIG. 11a) and the properties of the AP as measured by upstroke velocity (dV/dtₘₐₓ), amplitude (APA), duration at 90 and 80% of repolarisation (APD 90/80) and maximum diastolic potential (MDP) as summarized in the Table 3. Pacemaker-like APs are characterized by prominence of phase 4 depolarization, slow upstroke velocity (dV/dtₘₐₓ), and a smaller APA. The ventricle-like APs can be distinguished by the presence of a significant plateau phase of the AP resulting in a long duration, and high upstroke velocity and APA. The atrial-like APs show a triangular shape with a short duration, and high upstroke velocity and APA. The Purkinje-like APs are characterized by the presence of a notch and plateau phase as well as high upstroke velocity. In addition, a subset of cells (n=19) showed an intermediate AP phenotype (FIG. 11b), which exhibits characteristics of ventricle- and pacemaker-like morphology at the same time (slow upstroke velocity, long duration; Table 3). Similar results are also found in ESC-derived cardiomyocytes. These results suggest that mouse SSCs are able to differentiate into a distinct subset of cardiac lineages, including conduction system myocytes (He, J. Q., et al., Circ Res 93, 2003, 32-9).

To determine the functional expression of α-adrenergic receptors in cardiomyocytes, the effects of β-adrenergic agonist isoproterenol (Iso; 1 μmol/L) on APs were studied. It is shown that the AP frequency was significantly increased from 0.80±0.15 Hz to 1.62±0.21 Hz (n=5; P<0.05) in response to Iso stimulation (FIG. 11c). These results demonstrate that β-adrenergic receptors are present in maGSC-derived cardiomycytes and stimulation of these receptors produces a positive chronotropic response. This is again in line with data from the ESC-derived cardiomyocytes. Furthermore, cadmium (0.5 mmol/L), a non-specific blocker of voltage-gated Na⁺ and L-type Ca²⁺ channels completely abolished spontaneous APs (FIG. 11d) proving that Na⁺ and Ca²⁺ channels critically contribute to the observed APs. A further hint for the involvement of fast depolarising Na⁺ channels are AP upstroke velocities in the magnitude of 30-50 Vs⁻¹ for ventricle-, purkinje and atrial-like APs (Table 3). Especially ventricle- and purkinje-like APs showed plateau phases obviously caused by Ca²⁺ channel currents.

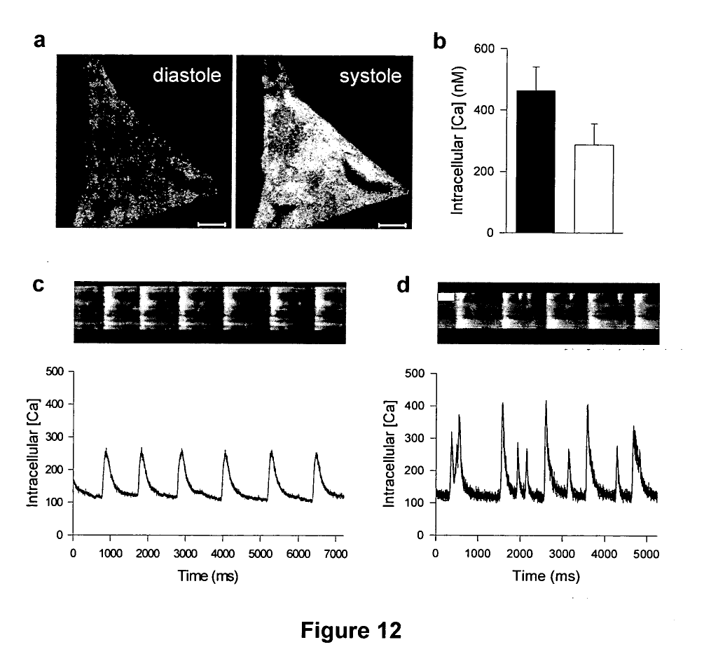

Intracellular Ca²⁺ was assessed in SSC-derived cardiomyocytes at day 5+7 using confocal microscopy. A typical triangle-shaped cardiomyocytes is presented in FIG. 12 a during low diastolic [Ca²⁺]ᵢ and high systolic [Ca²⁺]ᵢ. Calcium increased homogenously throughout the cell pointing to a fine regulated Ca²⁺ release from intracellular Ca²⁺ stores, most likely the sarcoplasmic reticulum (SR). The amplitudes measured in SSC-derived ventricle-like cardiomyocytes are similar to those measured in ESC-derived cardiomyocytes being 464±77 nM vs. 287±69 nM (FIG. 12b) also comparable to adult cardiac myocytes (Maier, L. S. et al. Circ Res 2003, 92, 904-11). Both are in the range of that measured in adult cardiac myocytes. Using line scan mode, rhythmic Ca²⁺ transients were found (FIG. 12c) and even small elementary Ca²⁺ release events (Ca²⁺ sparks; Cheng, H., et al., Science 1993, 262, 740-4). (FIG. 12d), which are mainly due to SR Ca²⁺ release through a cluster of ryanodine receptors (RyRs) appeared.

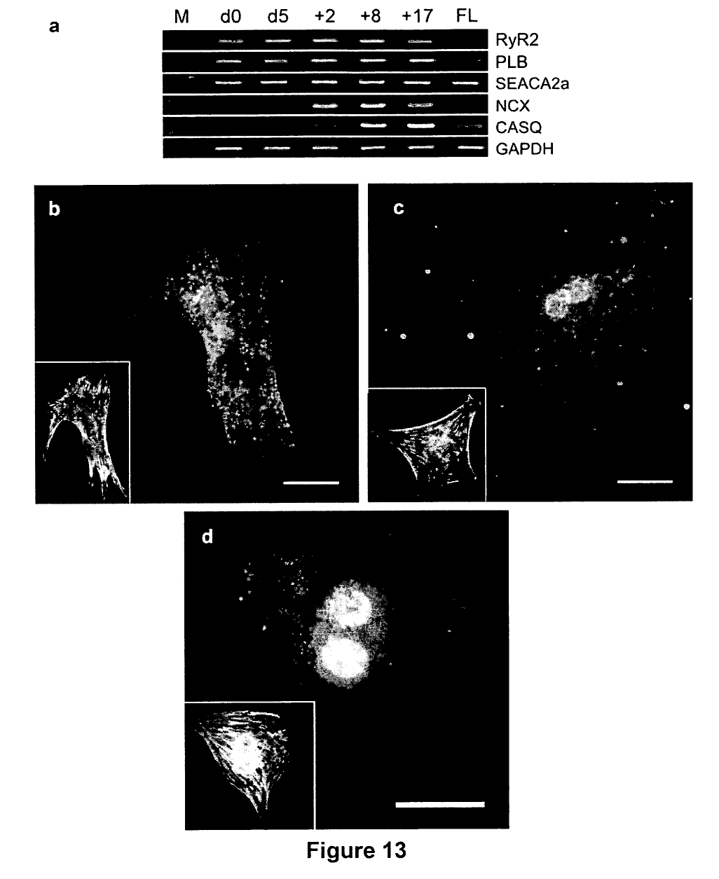

The cardiac RyR2 serves as the major SR calcium-release channel to mediate the rapid rise of cytosolic free calcium. We find that the gene encoding RyR2 is expressed during cardiac differentiation of mouse maGSCs (FIG. 13a). In addition, genes encoding phospholamban (PLB), SERCA2a isoform and NCX1 as well as calsequestrin (CASQ), a high capacity Ca²⁺-binding protein in the SR are expressed during cardiac differentiation of mouse maGSCs (FIG. 13a). This expression pattern is similar to those described for cardiac differentiation of ESCs. To study the distribution of RyR2 and Ca²⁺-handling proteins SERCA2a and NCX1 throughout the cardiomyocytes, we performed double staining in maGSC-derived cardiomyocytes at day 5+18. We found mainly a diffused and punctuated distribution of RyR2 in maGSC-derived cardiac cells (FIG. 13b). Of note, we observed partial organization of RyR2 in striated-like structures (FIG. 13b, arrow head), which is found in cardiomyocytes until postnatal day 6 when SR begins to organize. Immunocytochemical staining reveals that SERCA2a and NCX1 are mainly expressed in a fine granular network-like pattern throughout cardiomyocytes (FIG. 13c,d) comparable to those of neonatal cardiomyocytes. These results suggest that the organisation of the SR is not fully developed in maGSC-derived cardiac cells. This is also comparable with the ESC-derived cardiac cells at late developmental stages.

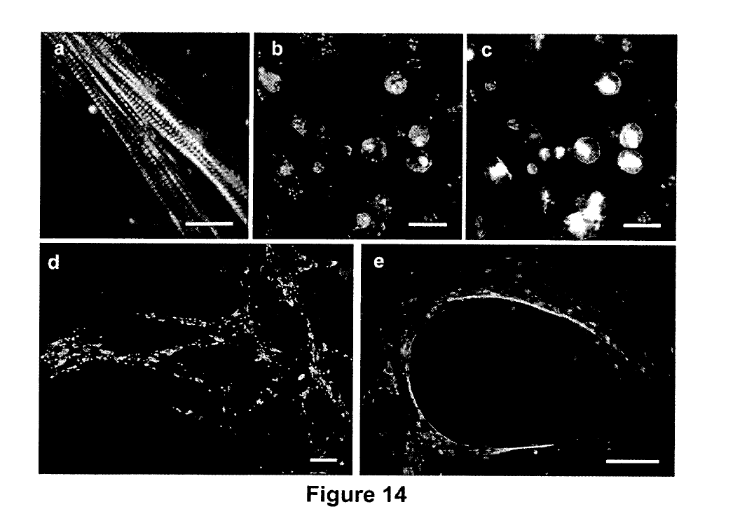

The differential expression of cardiac and skeletal muscle-specific transcription factors makes it possible to distinguish maGSC-derived cardiomyocytes from maGSC derived skeletal muscle cells. During EB differentiation, genes encoding the myogenic regulatory factors MyoD and Myf5 are strongly expressed from day 5+8 (FIG. 7) whereas cardiac-specific transcription factors are strongly expressed from an earlier stage (day 5). Moreover, the first myoblasts positive for nebulin appear at day 5+7, which is never seen in cardiac cells. The formation of multinucleated contracting myotubes positive for nebulin was only found at late stages (FIG. 14a). These data show that from early on the cardiac and skeletal muscle lineages are different.

Vascular cells (endothelial and smooth muscle cells), which are essential for maintaining cardiac integrity and function in vivo can also be differentiated spontaneously from maGSC-derived EBs. The genes encoding smooth muscle α-actin (SM-α-actin), vascular smooth muscle MHC (VSM-MHC), platelet/endothelial-cell adhesion molecule 1 (PECAM-1), vascular endothelial growth factor receptor 2 (also known as flk-1) and von Willebrand factor (vWF) were expressed during EB differentiation (FIG. 7). Rabbit polyclonal antibody against vWF (DAKO), and mouse monoclonal antibody against smooth muscle α-actin (clone 1A4, Sigma) were used for analysing vascular cell differentiation. In EB outgrowths we found cells double positive for DiI-conjugated acetylated low-density lipoproteins (ac-LDL-DiI) uptake (FIG. 14b) and lectin binding (FIG. 14c) characteristics for endothelial cells (Dimmeler, S. et al., J Clin Invest 108, 2001, 391-7). We also observed the formation of cellular networks and tube-like structures in EB outgrowths at later stages using antibodies against vWF (FIG. 14d) and SM-α-actin (FIG. 14e) indicating that these net-like and tubular structures of maGSC-derived cells consist of vascular endothelial and smooth muscle cells.

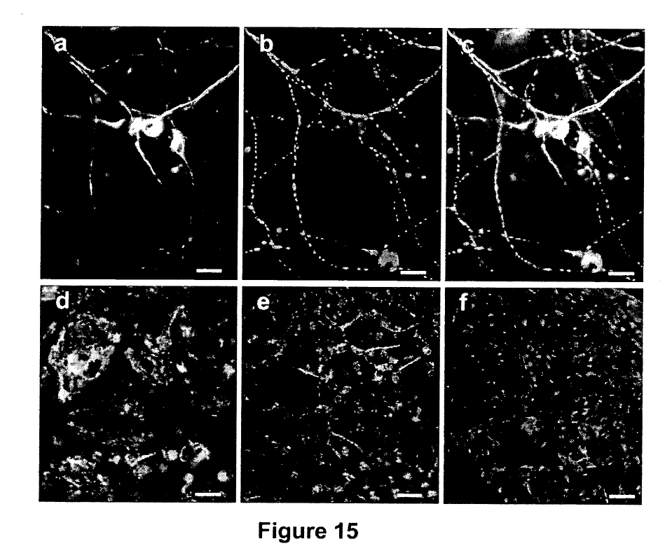

The neuroectoderm differentiation in the maGSC-derived EBs was confirmed by the gene expression of nestin, a marker for neuroepithelial precursors, and genes expressed in differentiated neurons, such as synaptophysin and TH (FIG. 7). Differentiated neural cells were analysed using antibodies against nestin (Rat401, DSHB), neurofilament polypeptide 160 kDa (NFM, NF-09, abcam), and tyrosine hydroxylase (TH, Chemico). We found a high level of nestin-positive neuroprogenitors at day 5+5 (data not shown). Although spontaneous differentiation efficiency of maGSCs into neuronal cells (FIG. 15a-c) was lower (only 25% of EBs containing neuronal cells at day 5+11) in comparison to cardiac differentiation (90% of EBs containing cardiomyocytes), maGSCs could differentiate into dopaminergic neurons under standard EB differentiation condition (FIG. 15a-c).

Epithelial-like cells found in all EB outgrowths are characterized using antibody anti-Pan cytokeratin (clone C-11, Sigma) which reacts with a wide variety of epithelial tissues (FIG. 15d). Cytokeratin 10 (CK10) which is present in keratinizing stratified epithelia was expressed during late differentiation of EBs (FIG. 7). It was also found the cells with phenotypic characteristics of hepatocytes in maGSC-derived EBs. The expression of the early (αFP) and late (CK18) markers of hepatocyte differentiation was found during EB differentiation (FIG. 7). At day 5+8, cells expressing αFP (DAKO) reveal two different morphologies: round or spindle-shaped cells. At day 5+17, many large epithelioid cells stained positive for CK18 (Ks18.04, Progen Biotechnik; FIG. 15e). In addition, many cells stained for CK7 (DAKO) were found, which serves as a maturation marker in bile duct cells (FIG. 15f).

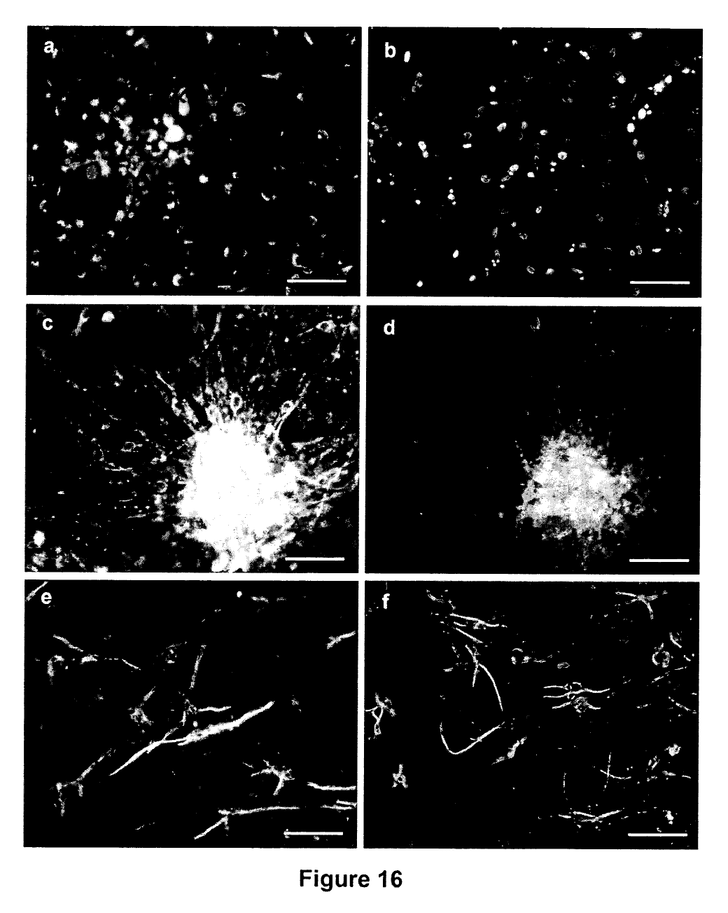

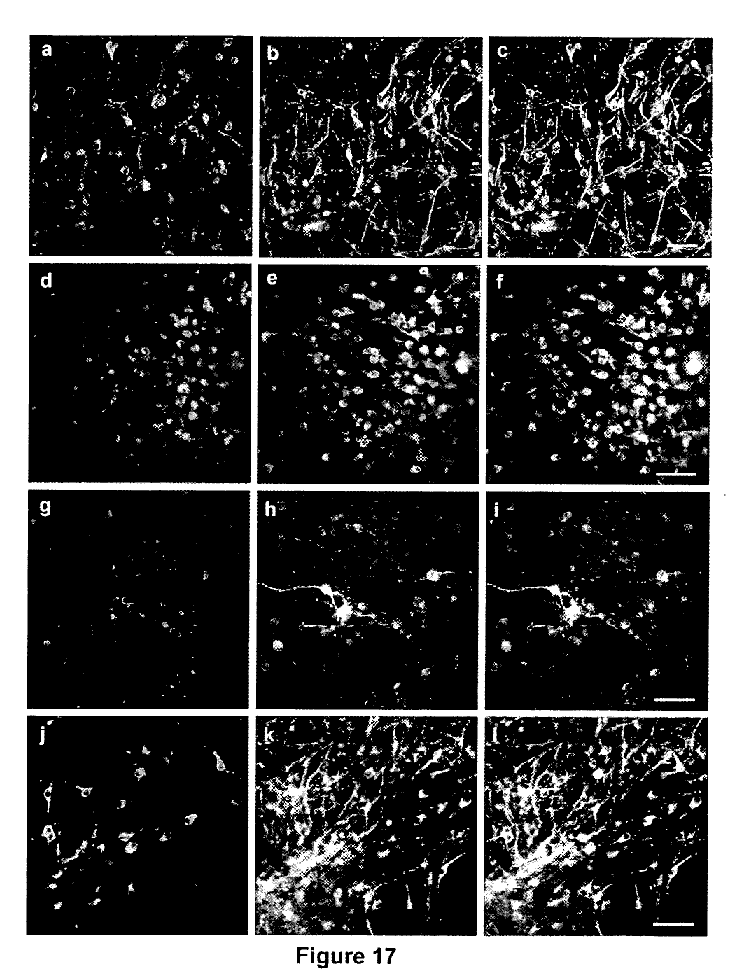

For lineage-selective differentiation of maGSCs into neural cells, neural precursor differentiation from maGSCs in serum-free adherent monoculture was first induced. To start monolayer differentiation, 3×10⁵ maGSCs in 3 ml standard ESC medium containing LIF were plated per 60 mm 0.1% gelatine-coated tissue culture dish (Nunc). Cells were maintained overnight, attached the culture dish and then switched to neural stem cell (NSC) medium (DMEM/F12 medium containing 2 mM glutamine, 1×N2 (Invitrogen), 1×NEAA, β-ME, 20 ng/ml bFGF and 10 ng/ml EGF) to start differentiation for 6 days. Medium is renewed every other day. Cells were then replated onto fresh tissue culture dishes in NSC medium containing 2% FCS, maintained overnight and then switch to NSC medium without FCS. After further cultivation for 4 days, cells were fixed and immunostained with antibodies against nestin (FIG. 16a) and Sox2 (clone 245610, R&D Systems; FIG. 16b). It was found that at this stage, over 90% of cells are positive for nestin (FIG. 16a) and Sox2 expression was reduced (FIG. 16b) indicating that cells lose pluripotent status and predominantly commit to a neural fate. Neuronal differentiation was initialed by resuspending the cells in N2B27-I medium (DMEM/F12 medium supplemented with 25 μg/ml insulin, 100 μg/ml apo-trasnferrin, 6 ng/ml progesterone, 16 μg/ml putrescine, 30 nM sodium selenite (all from Sigma) and 50 μg/ml bovine serum albumin fraction V, combined 1:1 with Neruobasal™ medium supplemented with 1×B27 (Invitrogen), 2 mM Glutamine and 1×β-ME) containing 20 ng/ml bFGF and replating onto fibronectin- or PDL-laminin-coated tissue culture dishes. Medium is renewed every 2-3 days. After 2 weeks, mature neurons could be detected in culture using antibodies against neuronal markers βIII-tubulin (TuJ1, BAbCO; FIG. 16c) and SNAP25 (Sigma; FIG. 16d). In addition, glial Fibrillary Acidic Protein)-positive astrocytes (GFAP; using monoclonal antibody G-A-5 anti-GFAP; Sigma; FIG. 16e) and O1-positive oligodendrocytes (oligodendrocyte marker, monoclonal antibody 59, Chemicon; FIG. 16f) can also be detected. Further analyses show that mouse maGSCs are able to differentiate into a distinct subset of neuronal lineages, including serotonin-producing (using polyclonal antibody anti-serotonin, Sigma; FIG. 17a-c), glutamic acid decarboxylase 65/67)-positive GABAergic (GAD65/67; using polyclonal antibody anti-GAD65/67, Sigma; FIG. 17d-f), glutamate transporter EAAC1-positive (using polyclonal antibody anti-EAAC1, Chemicon; FIG. 17g-i) and TH-positive dopaminergic neurons (FIG. 17j-l).

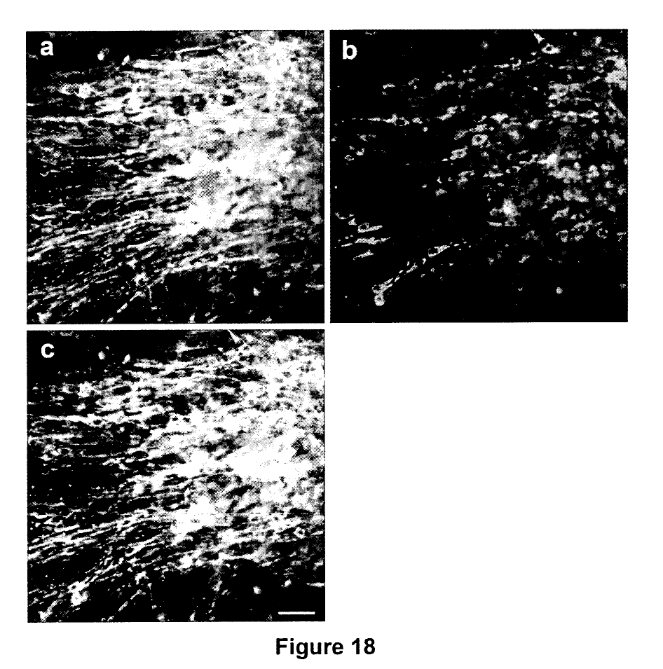

To induce the generation of TH-positive cells, cells were resuspented in N2B27-I medium containing 20 ng/ml bFGF, 400 ng/ml sonic hedgehog (R&D) and 100 ng/ml FGF8 (R&D) and replating onto PDL-laminin-coated tissue culture dishes. Two days later, medium is changed to N2B27-I plus 20 ng/ml bFGF. Medium is then renewed every 2-3 days. High number of TH-positive cells among βIII-tublin-positive neurons were detected (FIG. 18).

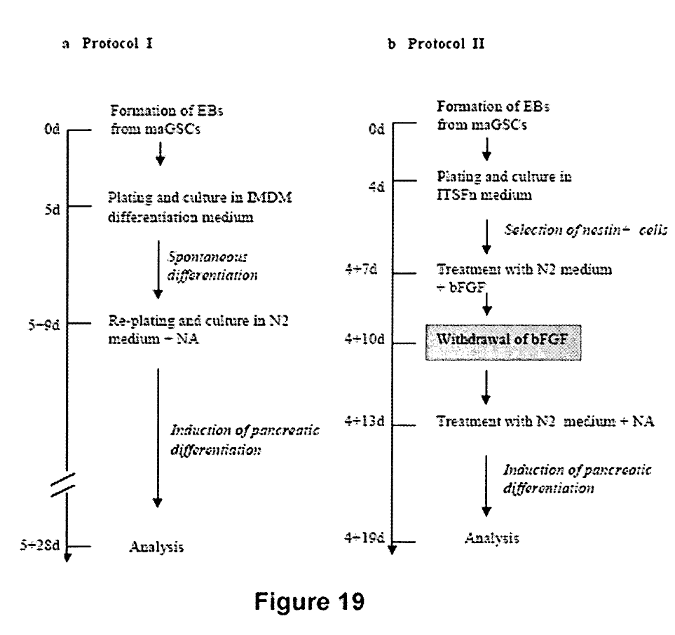

To induce pancreatic differentiation of maGSCs, two protocols used for ESC differentiation were applied. The differentiation protocol I was adapted from Blyszczuk et al. (FIG. 19a). EBs were cultured in IMDM differentiation medium for 9 days after plating at day 5 (=5+9 days) and the medium was changed every two days. At day 5+9 the EBs were dissociated with 0.1% trypsin/0.08% EDTA in PBS (1:1) into small clusters and replated onto 6-cm culture dishes for RT-PCR analysis or onto 6-cm culture dishes containing coverslips for immunofluorescence analysis. All culture dishes had been coated with 0.01% poly-L-ornithine (Sigma) and with 1 μg/ml laminin (Sigma). Treatment with poly-L-ornithine and laminin was done successively overnight at 37° C. with PBS washing steps in between. The dissociated clusters were cultured in N2 medium supplemented with 15% FCS and 10 mM nicotinamide (Sigma). The N2 medium consisted of DMEM/F12 (1:1; Invitrogen) supplemented with 20 nM progesterone, 100 μM putrescine, 1 μg/ml laminin, 30 nM sodium selenite, 50 μg/ml transferrin, 5 μg/ml fibronectin (all from Sigma), 25 μg/ml insulin and 1×B27 media supplement (both from Invitrogen). At day 5+10, FCS was removed and afterwards the medium was changed every three days. The experiment was finished at day 5+28.

The differentiation protocol II was adapted from Lumelsky et al. (FIG. 19b). At day 4, EBs were plated onto gelatinzed 6-cm culture dishes in ITSFn medium consisting of DMEM/F12 (1:1; Invitrogen) supplemented with 5 μg/ml insulin (Invitrogen), 50 μg/ml transferrin, 30 nM sodium selenite and 5 μg/ml fibronectin (all from Sigma). The medium was changed every three days. At day 4+7, the cells were treated with N2 medium supplemented with 10 ng/ml bFGF. At day 4+10, bFGF was withdrawn and at day 4+13, 10 mM nicotinamide (NA) was added to the N2 culture medium. Cells were grown in this medium until day 4+19, when the experiment was finished.

For protocol I, 5-day-old EBs were plated in IMDM differentiation medium and EB outgrowths were dissociated into small clusters and replated at day 5+9 when cardiomyocytes, smooth muscle cells, endothelial and epithelial cells were detected according to their characteristic morphology (data not shown). Cell clusters were then cultured in pancreatic differentiation medium (serum-free N2 medium supplemented with NA). For protocol II, 4-day-old EBs were plated in ITSFn medium and nestin-positive cells were selected in serum-free medium.

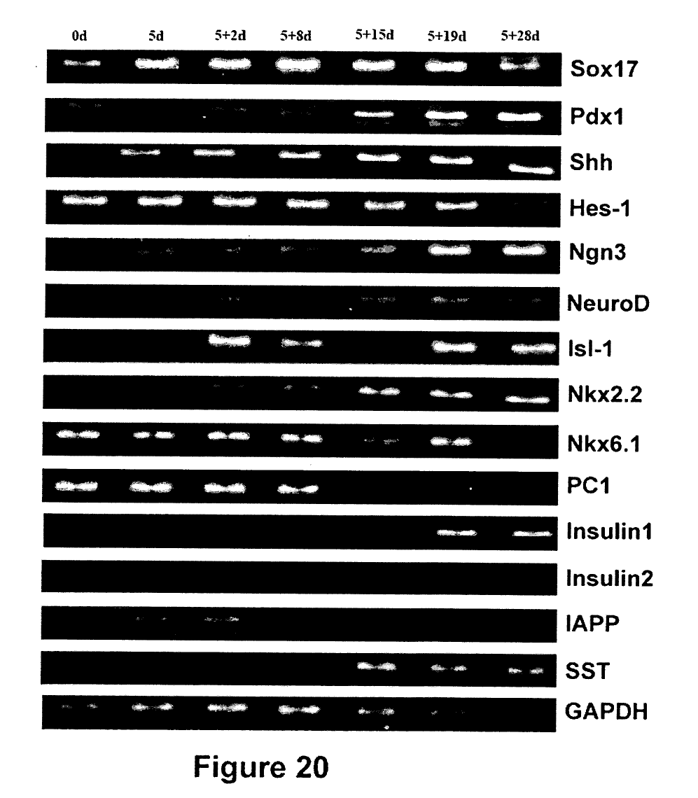

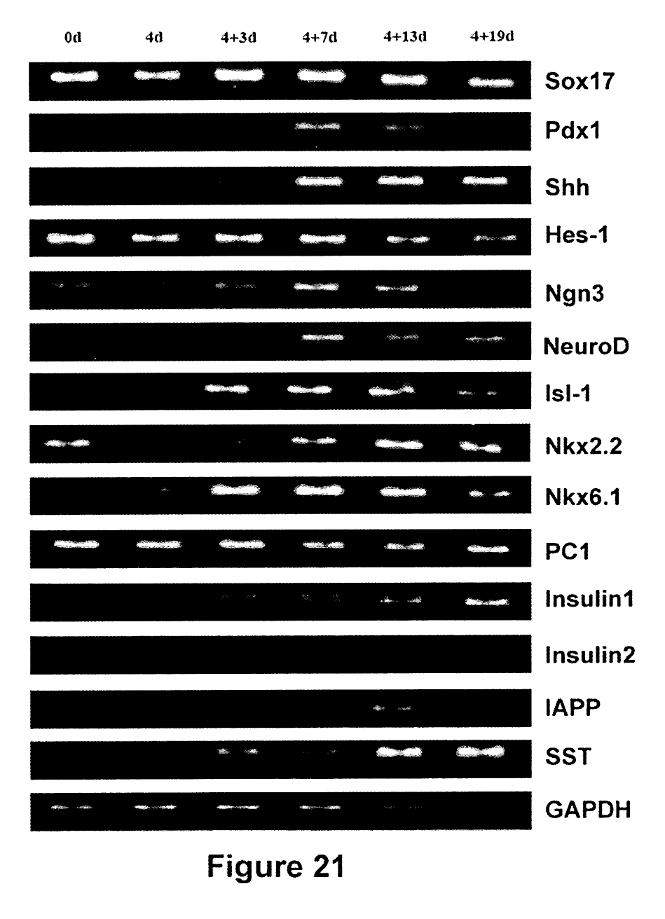

RT-PCR analyses showed that genes encoding transcription factors that exert important functions in the control of pancreatic differentiation as well as genes encoding proteins that are normally present in pancreas were expressed in a developmentally controlled pattern (FIGS. 20, 21). Sox17, a transcription factor that is considered as an early endoderm marker, was detected in both protocols in all samples. Pdx1, a key transcription factor is generally described to be expressed at elevated levels in early pancreatic development and in the mature pancreas where it positively regulates insulin expression. In the endocrine cell precursors it is transiently down-regulated. A comparable pattern was detected in samples cultured according to both protocol I (FIG. 20) and protocol II (FIG. 21). For the pancreas to develop, Sonic hedgehog (Shh) ought to be repressed. This repression could not be seen in this study probably because RNA from all cells of a culture dish was isolated and analyzed. This means that other cell types than developing pancreatic cells were present which did not show SHH repression. Hes1, a basics helix-loop-helix (bHLH) protein represses bHLH transcription factors like neurogenin 3 (Ngn3). Hes1 keeps cells at a precursor state and inhibits differentiation. In this study, expression of Hes1 was slightly down-regulated in both protocols indicating a certain balance between differentiation and selfrenewal that prevents depletion of the pool of progenitor cells. The up-regulation of Ngn3 in both protocols is also in accordance with the early findings that Ngn3 is a marker for the endocrine pancreas precursor. Another factor, that is involved in islet cell differentiation as well as neural development is NeuroD (=β2). Expression of the NeuroD gene was detected in both protocols. Isl-1, a transcription factor that binds to the enhancer region of the insulin gene, Nkx6.1 and Nkx2.2, the latter being a β-cell competence factor, were expressed from early stages on. Proprotein convertase 1 (PC1), the enzyme that processes proinsulin to insulin, was expressed throughout the differentiation. Insulin 1 was first detected in protocol I at day 5+19 and up-regulated as differentiation proceeded. In protocol II, insulin 1 was detected already at day 4+3 and kept at a relatively constant level as differentiation proceeded. In both protocols, insulin 2 could only be detected in the terminal stage at a very low level. Islet amyloid polypeptide (IAPP) was up-regulated in progenitor cells. During EB differentiation, somatostatin (SST), which is produced by δ cells, was up regulated showing that differentiation of other cell types of the islet of Langerhans was also induced.

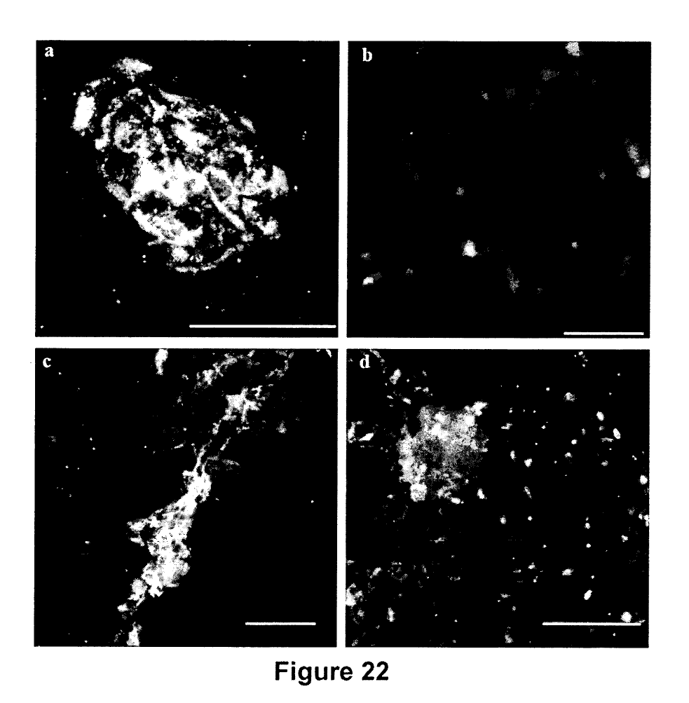

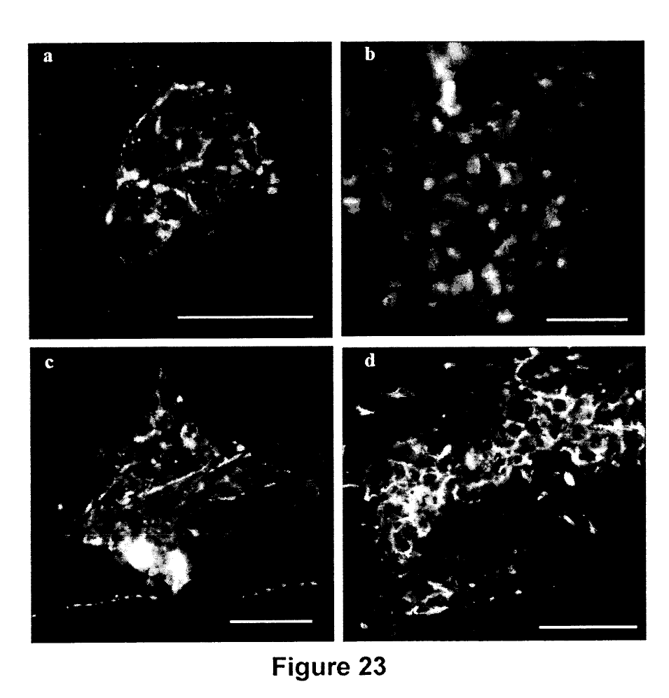

To answer the question whether nestin is involved in pancreatic differentiation of maGSCs in vitro, co-localization of nestin and Pdx1 (using polyclonal antibody against Pdx1, Absam; FIG. 22) as well as nestin and C-peptide (using polyclonal antibody against C-peptide, Linco; FIG. 23) was analyzed by immunofluorescence at early and late stages of differentiation. Expression of Pdx1 was detected at both stages in both protocols in accordance with its key role in early progenitors and in mature pancreas. A high co-expression of nestin and Pdx1 was found at early stages in both protocols (FIG. 22a, c). Continued differentiation up to late stages resulted in most Pdx1-expressing cells being negative for nestin in both protocols (FIG. 22b, d). However, at the late stage, only a small fraction of cells were Pdx1-positive (FIG. 22c) indicating the low efficiency of pancreatic differentiation. Expression of C-peptide was detected at both early and late stages in both protocols (FIG. 23). C-peptide is a by-product of insulin-production. Co-expression of nestin and C-peptide was analyzed, too. Double-positive cells at an early differentiation stage occurred (FIG. 23a, c). However, at early stage, most nestin-positive cells stained negative for C-peptide (data not shown). At intermediate (FIG. 23b) and late (FIG. 23d) stages, nestin-expression was less distinct while many cells were C-peptide-positive. The decreased co-expression of nestin and Pdx1 as well as nestin and C-peptide during maGSC-derived pancreatic differentiation suggests that nestin is transiently involved in pancreatic differentiation in vitro. In addition, at late stages in both protocols many nestin-positive cells were negative for Pdx1 or C-peptide and showed neuron-like morphology (data not shown).

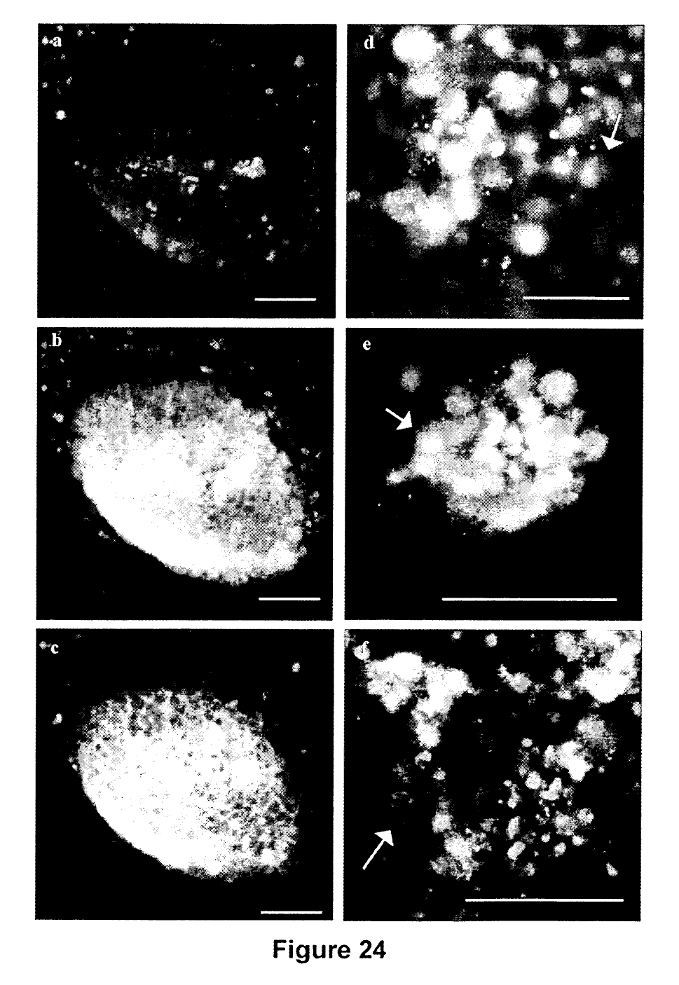

To investigate whether pancreatic hormone-producing cells could be generated from mouse maGSCs, immunofluorescence analyses were performed. At the terminal differentiation stage in protocol I, insulin-expressing islet-like clusters (using polyclonal antibody against insulin, Dako) could be demonstrated (FIG. 24a). These insulin-positive cells were also stained for C-peptide (FIG. 24b, c) providing evidence for endogenous insulin-production. Clusters co-expressing insulin and C-peptide were also detected in protocol II (data not shown).

Furthermore, cells expressing glucagon (using polyclonal antibody against glucagon, Dako), somatostatin (using polyclonal antibody against somatostatin, Dako) and pancreatic polypeptide (using polyclonal antibody against pancreatic polypeptide, Linco; FIG. 24d-f) were detected at the terminal differentiation stage indicating that other cell types of the endocrine pancreas could be generated from maGSCs in vitro.

Action Potential Measurements in Cardiomyocytes Derived from Mouse EBs:

The membrane potential (Em) of cardiomyocytes was measured at room temperature with ruptured-patch whole cell current clamp (Maier, L. S., et al., Circ Res 2003, 92, 904-11). Fire-polished glass microelectrodes of >10 MΩ resistance when filled with pipette solution were used. Pipette solution contained (in mM) 120 potassium aspartate, 8 KCl, 7 NaCl, 1 MgCl₂, 10 HEPES, 5 Mg-ATP, 0.3 Li-GTP (pH 7.2 with KOH). A normal Tyrode's solution with 2 mM CaCl₂ served as bath solution. Access resistance was typically ˜20 MΩ after patch rupture. Liquid junction potentials were corrected before recording started. Spontaneous action potentials (APs) were recorded 1 min after patch rupture and followed for about 2 min. Signals were acquired at 1 kHz, filtered with 2.9 and 10 kHz Bessel filters, and recorded with an EPC10 amplifier (HEKA Elektronik, Lambrecht/Pfalz, Germany). Action potentials were averaged from 10 single APs and analysis was done to determine the maximum rate of rise of the AP upstroke (dV/dtmax), AP amplitude (APA), AP duration at 90% of repolarisation (APD 90) and the maximum diastolic potential (MDP).

### Intracellular Calcium Measurements Using Confocal Laser Microscopy.

Intracellular calcium ([Ca²⁺]ᵢ) signals were recorded after incubating cells with 10 μM fluo-4 acetoxymethylester (Mobitec) for 15 min on a laser scanning confocal microscope (Zeiss LSM 5 PASCAL) (Maier, L. S., et al., Circ Res 2003, 92, 904-11). Cells were washed with Tyrode's solution as described above. Fluo-4 was excited via an argon laser (488 nm; 30 mW) and emitted fluorescence (F) was collected through a 505 nm long-pass emission filter. Changes in fluo-4 fluorescence (indicating fluctuation in cytosolic Ca²⁺) were recorded in frame and line scan mode while the cells were beating spontaneously. The images were acquired and analyzed using Zeiss software and fluorescence signals were normalized to basal cell fluorescence after fluo-4 loading (F₀). Intracellular Ca²⁺ was assessed using line scan modus and calibrated by the following pseudo-ratio equation [Ca]ᵢ=K_{d}(F/F₀)/(K_{d}/[Ca]ᵢ₋ᵣₑₛₜ+1−F/F₀) with K_{d}=1100 nM and [Ca]ᵢ₋ᵣₑₛₜ=100 nM.

5.2. Formation of EBs from Non-Human Primate and Human SSCs In Vitro

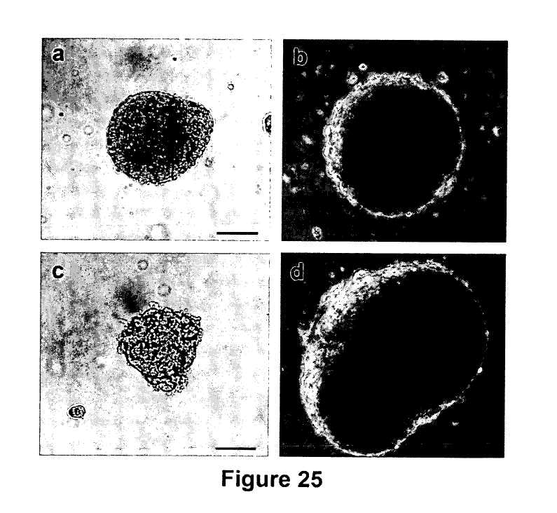

For differentiation in vitro, non-human primate and human SSCs were first cultivated as embryoid bodies (EBs) using hanging drop method as described for mouse maGSCs. However, we found that this method is not suitable for EB formation of non-human primate and human SSCs because of their slow proliferation rate. When the non-human primate and human SSCs were cultivated as mass culture in IMDM supplemented with 20% FCS, 2 mM L-glutamine, 1×NEAA and 50 μM β-ME, 3D aggregates were formed (FIG. 25). Briefly, non-human primate or human SSCs (n=500,000) in 5 ml of differentiation medium were cultured in bacteriological petri dishes for 2-8 days. It was found that the size of EBs derived from non-human primate SSCs at day 3 (FIG. 25a) is comparable with the size of mouse EBs (formed by hanging drop methods) at day 2. The EBs derived from human SSCs are rather smaller and looser (FIG. 25c).

6. In Vivo Differentiation of Murine maGSCs

### 6.1. Teratoma Formation in Immunodeficient Mice

To confirm the pluripotency of maGSCs in vivo, maGSCs (2×10⁶ cells per injection) were subcutaneously injected into 8-week-old male SCID-beige mice (ten mice total) for teratoma formation. Six weeks after injection the mice were sacrificed and the resulting teratomas were examined histologically.

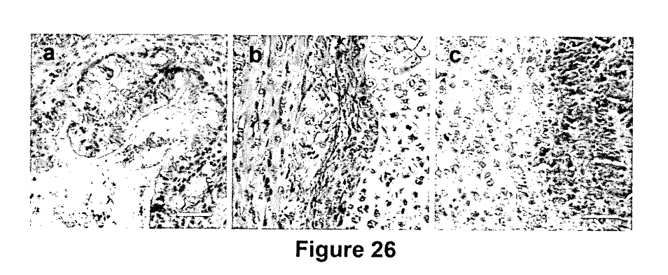

Results: The transplanted cells formed mature teratomas in all recipients (ten of ten) by 6 weeks after inoculation. The teratomas contained derivatives of three embryonic germ layers, including epithelium with intestinal differentiation (endoderm), striated muscle, smooth muscle, fat, bone and cartilage (mesoderm); and neural tissue (ectoderm) (FIG. 26a-c).

### 6.2. Chimaera Formation

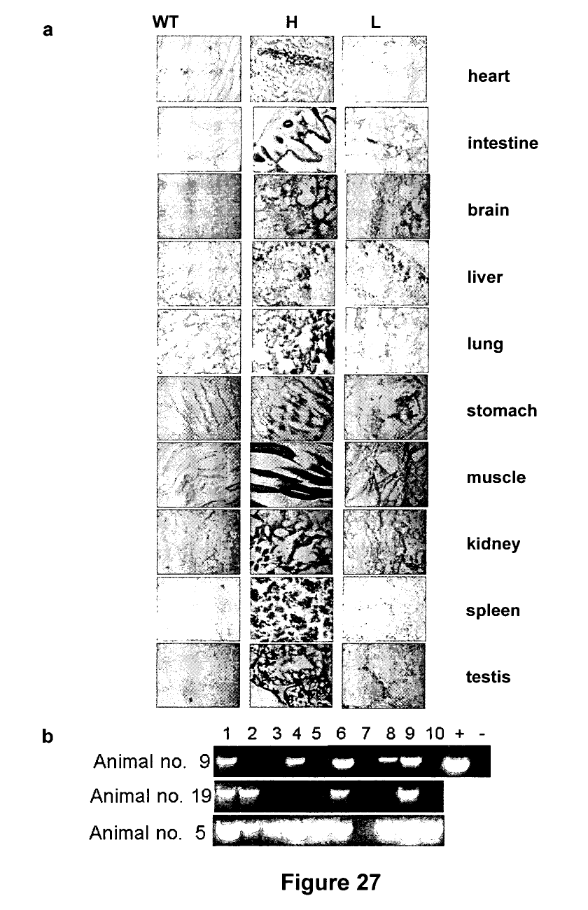

To determine the developmental potential of SSCs, SSCs were injected into early blastocysts. Wild-type blastocysts (3.5 dpc) were collected from the uteri of superovulated females by flushing with M2 medium (Sigma-Aldrich). Superovulation was induced by injection of 7.5 IU pregnant mare serum gonadotropin followed by injection of 7.5 IU human chorionic gonadotropin after a 48-hour interval. The collected blastocysts were washed with and cultivated in M16 (Sigma-Aldrich) under 5% CO₂ in air at 39° C. Blastocyst injection was carried out by injecting GFP+ cells using standard procedures (Kimura, Y. and Yanagimachi, R., Development 1995, 121, 2397-405). 10-15 SSCs from Stra8-EGFP/Rosa26 mice were microinjected into 3.5-day-old blastocysts of C57BL/6 mice. Sixty-five injected blastocysts were transferred into the uterus of pseudopregnant mice. The number of litters born (42 animals) and animals per litter (6-7) were in line with the birth rate seen with ESCs (Schoonjans, L. et al., Stem Cells 21, 2003, 90-7). Animals born from microinjected blastocysts were of similar size as normal animals and did not display overt abnormalities. Chimeras were identified by extraction of genomic DNA from tail biopsies and subsequent PCR analysis using LacZ specific primers. Chimaerism could be detected in 39 of 42 of mice (˜93%) by LacZ PCR of 4-week-old animals. LacZ PCR analysis of genomic DNA isolated from different tissues of three negative animals revealed LacZ positive in some tissues (data not shown) indicating that in these animals SSCs also contributed to some somatic tissues. Chimaeric animals that had LacZ+ cells, as determined by PCR in the tail clip analysis, were killed at 10-20 weeks. A contribution of LacZ+SSCs to many tissues, including heart, brain, intestine, lung, skeletal muscle, liver, kidney, spleen, and testis was found by PCR analysis (FIG. 27b). For LacZ staining, 10 μm frozen sections were stained in X-Gal solution containing 40 mM HEPES, pH7.4, 5 mM K3(Fe(CN)₆), 5 mM K4(Fe(CN)₆), 2 mM MgCl₂, 15 mM NaCl, and 1 mg/ml β-gal. Counterstaining was done with Red Fast. We found LacZ-expressing cells in heart, intestine, brain, liver, lung, stomach, muscle, kidney, spleen and testis of mice with varying degrees of chimaerism using LacZ staining (FIG. 27a).

### Prophetic Example

### SSCs in Cell-Based Organ Regeneration Therapy

Human SSCs or maGSCs derived from SSCs isolated from testicular biopsies may have great potential for cell-based organ regeneration therapy. It is believed that these cells are pluripotent and are able to differentiate into derivatives of all three embryonic germ layers. The SSCs as well as SSC-derived differentiated cells might be used for cell-based organ regeneration therapy. Although SSCs cells are pluripotent like ESCs, use of these cells as well as their derivatives for transplantation will allow establishment of individual cell-based therapy, because the donor and recipient can be identical. If immunological profile of these cells allows allogenic transplantation needs to be explored. Furthermore, the ethical problem associated with human ESCs is avoided. In addition to cell transplantation strategies, our discovery provides new possibilities to study diseases. The establishment of maGSCs from transgenic animals would allow developing various cell types in vitro for studying physiological or pathophysiological processes. Moreover, the establishment of cell lines from humans with genetic abnormalities or varieties would provide new options for studying the corresponding cellular phenotype under in vitro condition.

1. A method of producing embryonic stem cell (ESC)-like cells derived from adult mammalian testis, comprising the steps of:
(a) propagating isolated mammalian testicular cells in a suitable culture medium selected from the group consisting of
(i) a medium containing serum or serum replacement and optionally containing one or more growth factors selected from the group consisting of leukaemia inhibitory factor (LIF), glial cell line-derived neurotrophic factor (GDNF), basic fibroblast growth factor (bFGF), B27, and (2′Z,3′E)-6-Bromoindirubin-3′-oxime (BIO), and (ii) a medium containing serum or serum replacement on a suitable feeder layer; or any combination thereof for a period sufficient to allow the formation of ESC-like cells; and
(b) optionally obtaining the ESC-like cells from the culture.

2. The method of claim 1, wherein at most 50% of the ESC-like cells exhibit the c-kit antigen.

3. The method of claim 1, wherein mammalian testis cells are selected from the group consisting of human, non-human primate, mouse, rat, bovine, pig, goat, rabbit, ovine, horse, dog, cat and guinea pig cells.

4. The method of claim 4, wherein the mammalian cells are human or non-human primate cells.

5. The method of claim 4, wherein in step a) of claim 1 the medium is DMEM/F12 containing serum or serum replacement.

6. The method of claim 4, wherein in step a)(i) of claim 1 the medium additionally contains one or more of the growth factors selected from the group consisting of GDNF, LIF, bFGF, B27, and BIO.

7. The method of claim 6, wherein the propagating takes place on a suitable feeder layer.

8. The method of claim 1, wherein the mammalian cells are non-human primate cells, and the medium in step a) of claim 1 is DMEM further containing serum or serum replacement, and wherein the propagating takes place in the presence of an agent selected from the group consisting of LIF and a feeder layer, or any combination thereof.

9. The method of claim 1, wherein the mammalian cells are murine cells.

10. The method of claim 9, wherein in step a) of claim 1 the medium is DMEM containing serum or serum replacement.

11. The method of claim 9, wherein the propagating takes place in the presence of an agent selected from the group consisting of LIF and a feeder layer, or any combination thereof.

12. The method of claim 9, wherein the propagating takes place in the substantial absence of a factor selected from the group consisting of bFGF, EGF, and GDNF or any combination thereof.

13. The method of claim 1, wherein the propagating takes place in the substantial absence of a factor selected from the group consisting of steel factor (SF) and Bone Morphogenic Protein 8 (BMP8), or any combination thereof.

14. The method of claim 1, wherein prior to step a) of claim 1 the isolated mammalian testicular cells are propagated in a suitable cell culture medium which contains serum or serum replacement for 1 to 14 days.

15. The method of claim 1, wherein after step (a) and before step (b) the ESC-like cells are cultured in the presence of an agent selected from the group consisting of a suitable feeder layer and one or more suitable growth factors, or any combination thereof, for a period of 15-250.

16. The method of claim 1, wherein the isolated testicular cells are provided as a preparation derived from testis and enriched for spermatogonial stem cells (SSCs).

17. The method of claim 1, wherein the ESC-like cells express a product selected from the group consisting of Stra8 and the antigen Thy-1 or any combination thereof.

18. The method of claim 1, wherein the ESC-like cells are capable of forming embryoid bodies.

19. An ESC-like cell as obtainable by a process as described in claim 1.

20. The cell of claim 19 expressing Stra8.

21. A pharmaceutical preparation comprising ESC-like cells in accordance with claim 19 and a pharmaceutically acceptable carrier.

22. A method of producing embryoid bodies from ESC-like cells, comprising culturing the ESC-like cells according to claim 19 under conditions effective to yield embryoid bodies, and optionally recovering the embryoid bodies from the culture.

23. The method of claim 22, wherein the conditions effective to yield embryoid bodies are provided by a method selected from the group consisting of the hanging drop technique, mass culture, the methylcellulose technique, and the spinner culture method.

24. An embryoid body as obtainable by a process as described in claim 22.

25. A pharmaceutical preparation comprising an embryoid body in accordance with claim 24 and a pharmaceutically acceptable carrier.

26. A method of producing a tissue and a differentiated cell, comprising culturing an ESC-like cell according to claim 19 under conditions effective to yield the tissue or the differentiated cell, and optionally recovering the tissue or cell.

27. The method of claim 26, wherein the tissue is selected from the group consisting of heart, vessels, neurons, pancreas, skin, the eye, the nose, the ear, the spinal cord, a nerve, the trachea, the mouth, the esophagus, the small intestine, the large intestines, the ureter, the bladder, the urethra, a gland such as hypothalamus, pituitary, thyroid, and adrenal glands, the ovary, the oviduct, the uterus, the vagina, a mammary gland, the testes, the penis, a lymph nodes, a tendon, a ligament, brain, intestine, lung, muscle, stomach, liver, kidney, spleen, fat, bone, cartilage and epithelium.

28. The method of claim 26, wherein the cell is selected from the group consisting of a cardiomyocyte, a skeletal muscle cell, a smooth muscle cell, an endothelial cell, a vascular cell, a vascular smooth muscle cell, a neural cell, a neuronal cell, in particular a neuron, more particularly a dopaminergic neuron, a cholinergic neuron, a GABAergic neuron, a serotoninergic neuron; a glial cell, a dendritic cell, a pancreatic cell, particularly a pancreatic hormone-producing cell, more particularly an insulin-expressing cell, a beta-cell, a glucagon-expressing cell, an alpha-cell, a somatostatin-expressing cell, a pancreatic polypeptide-expressing cell; a hepatocyte, a chondroicyte, a blastocyte, an astrocyte, a oligodendrocyte, a blood cells, a progenitor cell, a urogenital cell, a gastrointestinal cell, a glandular cell, an adiopocyte, an osteocyte, a microglia, an epithelial or epitheloid cell, and a bile duct cell.

29. A tissue or differentiated cell as obtainable in accordance with claim 26.

30. A pharmaceutical preparation comprising a cell or tissue in accordance with claim 29 and a pharmaceutically acceptable carrier.

31. A method for producing cell lines, tissues, organs, blastocysts, and transgenic animals using a product selected from the group consisting of an ESC-like cell according to claims 19 or 20, an embryoid body according to claim 24, a tissue or cell according to claim 29, and any of the pharmaceutical compositions of claims 21, 25 or 30.

32. Use of an ESC-like cell according to claims 19 or 20, an embryoid body according to claim 24, or a tissue or cell according to claim 29, or any of the pharmaceutical compositions of claims 21, 25 or 30, for the manufacture of a pharmaceutical composition for cell-based organ regeneration therapy, tissue-based organ regeneration therapy, and autologous or allogenic cell, tissue or organ transplantation.

33. A method for treating a disease in a patient, wherein an effective amount of product selected from the group consisting of an ESC-like cell according to claims 19 or 20, an embryoid body according to claim 24, or a tissue or cell according to claim 29, and any of the pharmaceutical compositions of claims 21, 25 or 30, is administered to the patient, and wherein the disease or disorder is selected from the group consisting of chronic heart disease, Parkinson's disease, diabetes, liver failure, infertility and end-stage kidney disease, traumatic spinal cord injury, Purkinje cell degeneration, neural degeneration, Alzheimer' disease, Lewy Body Dementia, multiple sclerosis, duchenne's muscular dystrophy, muscular dystrophy, heart failure, osteogenesis imperfecta, osteoporosis, cancer, an autoimmune disease, a neurodegenerative disease, a respiratory disease, a vascular disease, a trauma, burn, head trauma, spinal cord injury, stroke, myocardial infarction, arthrosis, Huntington's disease, Tourette's syndrome, amytrophic lateral sclerosis, Addison's disease, pituitary insufficiency, liver failure, inflammatory arthrophathy, neuropathic pain, blindness, hearing loss, arthritis, a bacterial infection, a viral infection, a sexually transmitted disease and a damage of the skin, the eye, the nose, the ear, the brain, the spinal cord, a nerve, the trachea, the lungs, the mouth, the esophagus, the stomach, the liver, the small intestines, the large intestines, the kidney, the ureter, the bladder, the urethra, a gland such as hypothalamus, pituitary, thyroid, pancreas and adrenal glands, the ovary, the oviduct, the uterus, the vagina, a mammary gland, the testes, the penis, a lymph node, a vessel, the heart, a blood vessel, a skeletal muscle, a smooth muscle, a bone, cartilage, a tendon and a ligament.

34. A method for treating a patient in need of cell-based organ regeneration therapy, tissue-based organ regeneration therapy, and autologous or allogenic cell, tissue or organ transplantation, wherein an effective amount of a product selected from the group consisting of an ESC-like cell according to claim 19, an embryoid body according to claim 24, a tissue or cell according to claim 29, and any of the pharmaceutical compositions of claims 21, 25 or 30 is administered to the patient.
